(19) United States
(12) Patent Application Publication
(10) Pub. No.: US 2019/0388428 A1
(43) Pub. Date: Dec. 26, 2019

(54) A Pharmaceutical Composition Comprising An Oxazine Derivative And Its Use In The Treatment Or Prevention Of Alzheimer's Disease

(71) Applicant: NOVARTIS AG, Basel (CH)

(72) Inventors: Miloud Achour, Basel (CH); Brumo Galli, Basel (CH); Edgar John, Basel (CH); Michael Juhnke, Basel (CH); Dragutin Knezic, Waltham, MA (US); Vishal Shamji Koradia, Basel (CH); Rita Ramos, Basel (CH)

(21) Appl. No.: 16/478,704

(22) PCT Filed: Jan. 18, 2018
(86) PCT No.: PCT/IB2018/050312

§ 371 Date: Jul. 17, 2019

(30) Foreign Application Priority Data
Jan. 20, 2017 (EP) 17152481.2

(51) Int. Cl. A61K 31/5355; A61K 31/496; A61K 9/48; A61K 47/26; A61K 47/38

(52) U.S. Cl. CPC A61K 31/5355; A61K 31/496; A61K 47/38; A61K 47/26; A61K 9/4825

(57) ABSTRACT

The present invention relates to a pharmaceutical composition comprising an oxazine derivative BACE-1 inhibitor, a process for the preparation thereof, and its use in the treatment or prevention of Alzheimer's disease.

### FIELD OF THE INVENTION

The present invention relates to an oral immediate release pharmaceutical composition comprising an oxazine, a process for the preparation thereof, and its use in the treatment or prevention of Alzheimer's disease.

### BACKGROUND OF THE INVENTION

Alzheimer's disease (AD) is one of the most prevalent neurological disorders worldwide and the most common and debilitating age-related condition, causing progressive amnesia, dementia, and ultimately global cognitive failure and death. Currently, the only pharmacological therapies available are symptomatic drugs such as cholinesterase inhibitors or other drugs used to control the secondary behavioral symptoms of AD. Investigational treatments targeting the AD pathogenic cascade include those intended to interfere with the production, accumulation, or toxic sequelae of amyloid-β (Aβ) species (Kramp V P, Herrling P, 2011). Strategies that target decreasing Aβ by: (1) enhancing the amyloid clearance with an active or passive immunotherapy against Aβ; (2) decreasing production through inhibition of Beta-site-APP cleaving enzyme-1 (BACE-1, an enzyme involved in the processing of the amyloid precursor protein (APP)), are of potential therapeutic value.

The compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, referred to herein as “Compound 1”, is an orally active BACE inhibitor, previously described in WO 2012/095469 A1, with an approximately 3-fold selectivity for BACE-1 over BACE-2 and no relevant off-target binding or activity. In terms of its physical properties, it is non-hygroscopic, poorly wettable and poorly soluble in water. The neat drug substance has low bulk density and poor flow.

In order to be effective as an oral pharmaceutical agent, a drug substance must reach the systemic circulation, preferably via the gastroinstestinal tract, and reach its therapeutic target. From oral ingestion to reaching the blood stream, oral dosage forms, specifically the solid oral dosage forms (e.g. capsules) need to undergo complex steps of disintegration, dispersion and dissolution in order to achieve absorption via the gastrointestinal tract. Once absorbed, a drug substance still has to pass through the intestinal wall and hepatic metabolism before reaching the systemic circulation. Poorly soluble pharmaceutical compounds are well known to pose significant challenges to pharmaceutical scientists trying to develop suitable oral dosage forms. Since Compound 1 is poorly wettable and poorly soluble in water and aqueous buffers at intestinal pH, it is expected to have a relatively poor dissolution profile, adversely affecting its bioavailability. Furthermore, low solubility may also lead to high variability in in vivo absorption of the compound (Amidon G L et al. 1995). When tested in an in vitro permeability assay (PAMPA), Compound 1 showed high permeability. Pharmaceutical compounds, such as Compound 1, displaying low solubility and high permeability are, in general, expected to have their in vivo absorption affected by food administration (Heimbach T et al. 2013). Such changes in in vivo absorption due to food intake necessitates special dosage instructions (for example, to be administered before or after food), thereby giving rise to patient compliance issues. Therefore, it is an object of the present invention to provide a pharmaceutical composition comprising Compound 1 which ensures sufficient and consistent in vivo bioavailability of Compound 1. A further object of the present invention is to provide a pharmaceutical composition comprising Compound 1 which ensures sufficient and consistent in vivo bioavailability of Compound 1 whilst minimising the potential for food mediated changes in absorption.

Micronization of neat drug substance, in order to increase the drug substance surface area and thereby improve its dissolution rate and bioavailability, was found to be extremely challenging at relevant operational conditions due to poor flow and the tendency of the drug substance to adhere to the mill. A further objective of the present invention is therefore to provide an improved milling method for Compound 1.

An experimental formulation (EF) of Compound 1 showed relatively poor bioavailability. The dissolution of a poorly wettable drug, and hence its bioavailability, may be improved, for example, by co-formulating with a surfactant. However, the levels of surfactant in the resultant pharmaceutical drug product must be tightly controlled and monitored over its shelf-life since surfactants are considered functional excipients. It is therefore a further object of the present invention to provide a pharmaceutical composition which improves the dissolution and bioavailability of Compound 1 without the use of surfactant.

It is also important that a pharmaceutical agent is chemically stable when formulated as a pharmaceutical composition. Preferably, the pharmaceutical agent is sufficiently stable such that refrigeration of the pharmaceutical composition is not required, to facilitate global transportation of the medicinal product and improve patient compliance. This aspect in particularly important in the context of the chronic dosing regimen anticipated for the treatment and prevention of Alzheimer's disease. It is therefore a further objective of the present invention to provide a pharmaceutical composition comprising Compound 1 wherein Compound 1 is sufficiently stable, preferably to a degree which avoids refrigeration of the pharmaceutical composition during long term storage in different climatic zones, for example as depicted in the ICH Q1A Guidance.

### SUMMARY OF THE INVENTION

During experimental development of the Compound 1 formulation, it was surprisingly found that the problem of poor relative bioavailability could be solved by manipulating the excipients and the porosity of the blend comprised within the pharmaceutical composition.

In a first aspect of the invention, there is therefore provided a pharmaceutical composition comprising the drug substance Compound 1 wherein subsequent to a single dose oral administration to a human subject the plasma Cmax value of the drug substance measured in ng/mL is a function of the drug substance dose in mg multiplied by a factor of 2.4, within a +/−range defined by the drug substance dose in mg multiplied by a factor of 0.7, when the pharmaceutical composition comprises greater than or equal to 10 mg of drug substance or less than or equal to 50 mg of drug substance.

In a second aspect of the invention, there is therefore provided a pharmaceutical composition comprising the drug substance Compound 1 and having a dissolution profile wherein at least 40% of the cumulative drug substance release is observed after 15 minutes dissolution testing using the basket apparatus method described in US Pharmacopeia Chapter <711> and the following testing parameters:
Dissolution medium: acetate buffer pH 4.5;
Apparatus 1: 100 rpm;
Total Measurement Time: 60 minutes; and
Temperature: 37±0.5° C.

In a third aspect of the invention, there is therefore provided a pharmaceutical composition comprising the drug substance Compound 1 and having a blend with:
(i) a median pore diameter of at least 1 μm, as determined by mercury porosimetry, within the 0.03 to 9 μm pore diameter range;
(ii) a cumulative pore volume of at least 200 mm³/g, as determined by mercury porosimetry, within the 0.03 to 9 μm pore diameter range; or
(iii) a cumulative pore volume of at least 600 mm³/g, as determined by mercury porosimetry, within the 0.004 to 130 μm pore diameter range.

During further experimental development of the Compound 1 formulation, it was surprisingly found that the problem of providing a sufficiently stable pharmaceutical composition comprising Compound 1 could be solved by formulating Compound 1 as described herein. In a fourth aspect of the invention, there is therefore provided a pharmaceutical composition comprising the drug substance Compound 1 wherein said drug substance is present within the pharmaceutical composition in an amount greater than 7% w/w.

In a fifth aspect of the invention, there is provided a pharmaceutical composition comprising Compound 1;
(i) a sugar alcohol;
(ii) starch or cellulose; and
(iii) hydroxypropyl cellulose or hydroxypropyl methylcellulose.

In a sixth aspect of the invention, there is provided a pharmaceutical composition according to the first, second, third, fourth or fifth aspect of the invention, for use in the treatment or prevention of Alzheimer's disease.

In a seventh aspect of the invention, there is provided a method for the treatment or prevention of Alzheimer's disease which method comprises administering to a patient the pharmaceutical composition according to the first, second, third, fourth or fifth aspect of the invention comprising a therapeutically effective amount of Compound 1.

In an eighth aspect of the invention, there is provided the use of a pharmaceutical composition according to the first, second, third, fourth or fifth aspect of the invention, for the treatment or prevention of Alzheimer's disease.

In a ninth aspect of the invention, there is provided the use of the drug substance Compound 1 for the manufacture of a pharmaceutical composition according to the first, second, third, fourth or fifth aspect of the invention, for the treatment or prevention of Alzheimer's disease.

During experimental development of the milling process, it was surprisingly found that poor flow and adherence of the drug substance to the mill could be overcome by co-milling with a sugar alcohol, such as mannitol.

In a tenth aspect of the invention, there is therefore provided a process for the preparation of a pharmaceutical composition comprising the drug substance Compound 1 wherein the drug substance is co-milled with a sugar alcohol.

### DESCRIPTION OF THE INVENTION

### List of Figures

FIG. 1 shows the X-ray powder diffraction pattern for crystalline Compound 1 (Form A) when measured using CuK_{α} radiation.

FIG. 2 shows the DSC thermogram for crystalline Compound 1 (Form A).

FIG. 3 shows the dissolution profile of the 25 mg capsule strength Compound 1 Experimental Formulation in various media.

FIG. 4 shows the dissolution profile of the 25 mg capsule strength Compound 1 Formulation A in various media.

FIG. 5 shows the dissolution profile of the 25 mg capsule strength Compound 1 Formulation B in various media.

FIG. 6 shows the dissolution profiles for 15, 25 and 50 mg Compound 1 dose strength Formulation B capsules (in pH 4.5 acetate buffer) FIG. 7 shows the dissolution profiles (in pH 4.5 acetate buffer) of 25 mg dose strength Formulation B capsules produced with blends of different median pore diameter and cumulative pore volume.

FIG. 8 shows the design of a human in vivo study to assess the relative bioavailability of formulations comprising Compound 1.

FIG. 9 shows the relative bioavailability of three different pharmaceutical compositions comprising Compound 1 in the human in vivo study described in FIG. 7.

FIG. 10 shows the design of a two part, open-label, two-period, fixed-sequence study in healthy subjects to evaluate the PK of Compound 1 when given alone and in combination with the strong CYP3A4 inhibitor itraconazole or the strong CYP3A4 inducer rifampicin.

### EMBODIMENTS OF THE FIRST ASPECT OF THE INVENTION

### Embodiment A1

A pharmaceutical composition comprising the drug substance Compound 1 wherein subsequent to single dose oral administration to a human subject the plasma Cmax value of the drug substance measured in ng/mL is a function of the drug substance dose in mg multiplied by a factor of 2.4, within a +/−range defined by the drug substance dose in mg multiplied by a factor of 0.7, when the pharmaceutical composition comprises greater than or equal to 10 mg of drug substance or less than or equal to 50 mg of drug substance.

### Embodiment A2

The pharmaceutical composition according to Embodiment A1, wherein the +/−range is defined by the drug substance dose in mg multiplied by a factor of 0.6, 0.5, 0.4, 0.3, 0.2 or 0.1.

### EMBODIMENTS OF THE SECOND ASPECT OF THE INVENTION

### Embodiment B1

A pharmaceutical composition comprising the drug substance Compound 1 having a dissolution profile wherein at least 40% of the cumulative drug substance release is observed after 15 minutes in dissolution testing using the basket apparatus method described in US Pharmacopeia Chapter <711> and the following testing parameters:
Dissolution medium: acetate buffer pH 4.5;
Apparatus 1: 100 rpm stirring;
Total Measurement Time: 60 minutes; and
Temperature: 37±0.5° C.

### Embodiment B2

The pharmaceutical composition according to Embodiment B1 wherein at least 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70% of the cumulative drug substance release is observed after 15 minutes.

### Embodiment B3

The pharmaceutical composition according to Embodiment B1 wherein at least 60% of the cumulative drug substance release is observed after 15 minutes.

### Embodiment B4

The pharmaceutical composition according to Embodiment B1 wherein at least 70% of the cumulative drug substance release is observed after 15 minutes.

### Embodiment B5

The pharmaceutical composition according to Embodiment B1 wherein at least 75% of the cumulative drug substance release is observed after 15 minutes.

### Embodiment B6

The pharmaceutical composition according to Embodiment B1 wherein at least 80% of the cumulative drug substance release is observed after 15 minutes.

### Embodiment B7

The pharmaceutical composition according to Embodiment B1 wherein at least 85% of the cumulative drug substance release is observed after 15 minutes.

### Embodiment B8

The pharmaceutical composition according to any one of Embodiments B1 to B7 wherein no more than 90, 91, 92, 93, 94, 95, 96, 97, 98 or 99% of the cumulative drug substance release is observed after 15 minutes.

### Embodiment B9

The pharmaceutical composition according to any one of Embodiments B1 to B7 wherein no more than 96% of the cumulative drug substance release is observed after 15 minutes.

### Embodiment B9

The pharmaceutical composition according to any one of Embodiments B1 to B7 wherein no more than 98% of the cumulative drug substance release is observed after 15 minutes.

### Embodiment B11

The pharmaceutical composition according to Embodiment B1 wherein 75%+/−20, 19, 18, 17, 16, 15, 14, 13, 12, 11, 10, 9, 8, 7, 6, 5, 4, 3, 2, or 1% of the cumulative drug substance release is observed after 10 minutes.

### Embodiment B12

The pharmaceutical composition according to Embodiment B1 wherein 75%+/−15% of the cumulative drug substance release is observed after 10 minutes.

### Embodiment B13

The pharmaceutical composition according to Embodiment B1 wherein 75%+/−10% of the cumulative drug substance release is observed after 10 minutes.

### Embodiment B14

The pharmaceutical composition according to Embodiment B1 wherein 75%+/−5% of the cumulative drug substance release is observed after 10 minutes.

### Embodiment B15

The pharmaceutical composition according to Embodiment B1 wherein 85%+/−13% of the cumulative drug substance release is observed after 15 minutes.

### Embodiment B16

The pharmaceutical composition according to Embodiment B1 wherein 85%+/−9% of the cumulative drug substance release is observed after 15 minutes.

### Embodiment B17

The pharmaceutical composition according to Embodiment B1 wherein 88%+/−5% of the cumulative drug substance release is observed after 15 minutes.

### Embodiment B18

The pharmaceutical composition according to Embodiment B1 wherein 79%+/−5% of the cumulative drug substance release is observed after 15 minutes.

### Embodiment B19

The pharmaceutical composition according to Embodiment B1 wherein 85%+/−7% of the cumulative drug substance release is observed after 15 minutes.

### Embodiment B20

The pharmaceutical composition according to Embodiment B1 wherein 90%+/−10% of the cumulative drug substance release is observed after 30 minutes.

### Embodiment B21

The pharmaceutical composition according to Embodiment B1 wherein 90%+/−8% of the cumulative drug substance release is observed after 30 minutes.

### Embodiment B22

The pharmaceutical composition according to Embodiment B1 wherein 85%+/−5% of the cumulative drug substance release is observed after 30 minutes.

### Embodiment B23

The pharmaceutical composition according to Embodiment B1 wherein 85%+/−2.5% of the cumulative drug substance release is observed after 30 minutes Embodiment B24: The pharmaceutical composition according to Embodiment B1 wherein 95%+/−5% of the cumulative drug substance release is observed after 30 minutes.

### Embodiment B25

The pharmaceutical composition according to Embodiment B1 wherein 95%+/−2.5% of the cumulative drug substance release is observed after 30 minutes.

### EMBODIMENTS OF THE THIRD ASPECT OF THE INVENTION

### Embodiment C1

A pharmaceutical composition comprising the drug substance Compound 1 and having a blend with a median pore diameter of at least 1 μm, as determined by mercury porosimetry, within the 0.03 to 9 μm pore diameter range.

### Embodiment C2

The pharmaceutical composition according to Embodiment C1 wherein the median pore diameter is at least 1.1, 1.2, 1.3, 1.4, 1.5, 1.6, 1.7, 1.8, 1.9, 2.0, 2.1, 2.2, 2.3, 2.4 or 2.5 μm within the 0.03 to 9 μm pore diameter range.

### Embodiment C3

The pharmaceutical composition according to Embodiment C1 wherein the median pore diameter is at least 1.4 μm within the 0.03 to 9 μm pore diameter range.

### Embodiment C4

The pharmaceutical composition according to Embodiment C1 wherein the median pore diameter is at least 1.8 μm within the 0.03 to 9 μm pore diameter range.

### Embodiment C5

The pharmaceutical composition according to any one of Embodiments C1 to C4 wherein the median pore diameter is less than 5, 4.5, 4, 3.5 or 3 μm within the 0.03 to 9 μm pore diameter range.

### Embodiment C6

The pharmaceutical composition according to any one of Embodiments C1 to C4 wherein the median pore diameter is less than 3 μm within the 0.03 to 9 μm pore diameter range.

### Embodiment C7

The pharmaceutical composition according to Embodiment C1 wherein the median pore diameter is 2 μm (+/−0.2 μm) within the 0.03 to 9 μm pore diameter range.

### Embodiment C8

A pharmaceutical composition comprising the drug substance Compound 1 and having a blend with a cumulative pore volume of at least 200 mm³/g, as determined by mercury porosimetry, within the 0.03 to 9 μm pore diameter range.

### Embodiment C9

The pharmaceutical composition according to Embodiment C8 comprising the drug substance Compound 1 wherein the cumulative pore volume is at least 205, 210, 215, 220, 225, 230, 235, 240, 245, 250, 255, 260, 265, 270, or 275 mm³/g within the 0.03 to 9 μm pore diameter range.

### Embodiment C10

The pharmaceutical composition according to Embodiment C8 comprising the drug substance Compound 1 wherein the cumulative pore volume is at least 250 mm³/g within the 0.03 to 9 μm pore diameter range.

### Embodiment C11

The pharmaceutical composition according to any one of Embodiments C8 to C10 comprising the drug substance Compound 1 and having a blend with a cumulative pore volume of less than 500, 450, 400, 350, 325 or 300 mm³/g within the 0.03 to 9 μm pore diameter range.

### Embodiment C12

The pharmaceutical composition according to any one of Embodiments C8 to C10 comprising the drug substance Compound 1 wherein the cumulative pore volume is less than 325 mm³/g within the 0.03 to 9 μm pore diameter range.

### Embodiment C13

The pharmaceutical composition according to Embodiment C8 having a blend with a cumulative pore volume of 200 mm³/g (+/−25 mm³/g) within the 0.03 to 9 μm pore diameter range.

### Embodiment C14

A pharmaceutical composition comprising the drug substance Compound 1 and having a blend with a cumulative pore volume of at least 600 mm³/g, as determined by mercury porosimetry, within the 0.004 to 130 μm pore diameter range.

### Embodiment C15

The pharmaceutical composition according to Embodiment C14 wherein the cumulative pore volume is at least 620, 640, 660, 680, 700, 720, 740, 760, or 780 mm³/g within the 0.004 to 130 μm pore diameter range.

### Embodiment C16

The pharmaceutical composition according to Embodiment C14 wherein the cumulative pore volume is at least 700 mm³/g within the 0.004 to 130 μm pore diameter range.

### Embodiment C17

The pharmaceutical composition according to any one of Embodiments C14 to C16 wherein the cumulative pore volume is less than 1500, 1400, 1300, 1200, 1100, 1000 or 975 mm³/g within the 0.004 to 130 μm pore diameter range.

### Embodiment C18

The pharmaceutical composition according to any one of Embodiments C14 to C16 wherein the cumulative pore volume is less than 1000 mm³/g within the 0.004 to 130 μm pore diameter range.

### Embodiment C19

The pharmaceutical composition according to Embodiment C14 wherein the cumulative pore volume is 800 mm³/g (+/−150 mm³/g) within the 0.004 to 130 μm pore diameter range.

### Embodiment C20

The pharmaceutical composition according to Embodiment C14 wherein the cumulative pore volume is 750 mm³/g (+/−100 mm³/g) within the 0.004 to 130 μm pore diameter range.

### Embodiment C21

The pharmaceutical composition according to Embodiment C14 wherein the cumulative pore volume is 750 mm³/g (+/−75 mm³/g) within the 0.004 to 130 μm pore diameter range.

### Embodiment C22

The pharmaceutical composition according to Embodiment C14 wherein the cumulative pore volume is 750 mm³/g (+/−50 mm³/g) within the 0.004 to 130 μm pore diameter range.

### EMBODIMENTS OF THE FOURTH ASPECT OF THE INVENTION

### Embodiment D1

A pharmaceutical composition comprising the drug substance Compound 1 wherein said drug substance is present within the pharmaceutical composition in an amount greater than 7% w/w.

### Embodiment D2

The pharmaceutical composition according to Embodiment D1 wherein the drug substance is present within the pharmaceutical composition in an amount greater than 7.1, 7.2, 7.3, 7.4, 7.5, 7.6, 7.7, 7.8, 7.9, 8.0, 8.1, or 8.2% w/w.

### Embodiment D3

The pharmaceutical composition according to Embodiment D1 wherein the drug substance is present within the pharmaceutical composition in an amount greater than 7.5% w/w.

### Embodiment D4

The pharmaceutical composition according to Embodiment D1 wherein the drug substance is present within the pharmaceutical composition in an amount greater than 8% w/w.

### Embodiment D5

The pharmaceutical composition according to any one of Embodiments D1 to D4 wherein the drug substance is present within the pharmaceutical composition in an amount less than 35% w/w

### Embodiment D6

The pharmaceutical composition according to Embodiment D1 comprising:
(i) 1 to less than 25 mg of drug substance Compound 1 wherein said drug substance is present within the pharmaceutical composition in an amount greater than 7% w/w; or
(ii) 25 to 50 mg of drug substance Compound 1 wherein said drug substance is present within the pharmaceutical composition in an amount greater than 17% w/w.

### Embodiment D7

The pharmaceutical composition according to Embodiment D1 comprising:
(i) 1 to less than 25 mg of drug substance Compound 1 wherein said drug substance is present within the pharmaceutical composition in an amount greater than 7.1, 7.2, 7.3, 7.4, 7.5, 7.6, 7.7, 7.8, 7.9, 8.0, 8.1 or 8.2% w/w; or
(ii) 25 to 50 mg of drug substance Compound 1 wherein said drug substance is present within the pharmaceutical composition in an amount greater than 17.2, 17.4, 17.6, 17.8, 18.0, 18.2, 18.4, 18.6, 18.8, 19.0, 19.2, 19.4, 19.6, 19.8, 20.0, 20.2, 20.4, 20.6 or 20.7% w/w.

### Embodiment D8

The pharmaceutical composition according to Embodiment D6 or D7 comprising:
(i) 1 to less than 25 mg of drug substance Compound 1 wherein said drug substance is present within the pharmaceutical composition in an amount less than 9, 10, 11, 12, 13, 14, 15, 16, 17, 18, 19, 20, 21, 22, 23, 24, 25, 26, 27, 28, 29, 30, 31, 32, 33, 34 or 35% w/w; or
(ii) 25 to 50 mg of drug substance Compound 1 wherein said drug substance is present within the pharmaceutical composition in an amount less than 21, 22, 23, 24, 25, 26, 27, 28, 29, 30, 31, 32, 33, 34 or 35% w/w.

### Embodiment D9

The pharmaceutical composition according to Embodiment D6 or D7 comprising:
(i) 1 to less than 25 mg of drug substance Compound 1 wherein said drug substance is present within the pharmaceutical composition in an amount less than 35% w/w; or
(ii) 25 to 50 mg of drug substance Compound 1 wherein said drug substance is present within the pharmaceutical composition in an amount less than 35% w/w.

### Embodiment D10

The pharmaceutical composition according to Embodiment D1 comprising:
(i) 1 to less than 25 mg of drug substance Compound 1 wherein said drug substance is present within the pharmaceutical composition in an amount between 7 and 35% w/w; or
(ii) 25 to 50 mg of drug substance Compound 1 wherein said drug substance is present within the pharmaceutical composition in an amount between 17 and 35% w/w.

### Embodiment D11

The pharmaceutical composition of Embodiment D1 comprising:
(i) 1 to less than 25 mg of drug substance Compound 1 wherein said drug substance is present within the pharmaceutical composition at 8.3% w/w+/−1%; or
(ii) 25 to 50 mg of drug substance Compound 1 wherein said drug substance is present within the pharmaceutical composition at 20.8% w/w+/−1%.

### Embodiment D12

The pharmaceutical composition of Embodiment D1 comprising:
(iii) 1 to less than 25 mg of drug substance Compound 1 wherein said drug substance is present within the pharmaceutical composition at 8.3% w/w+/−0.5%; or
(iv) 25 to 50 mg of drug substance Compound 1 wherein said drug substance is present within the pharmaceutical composition at 20.8% w/w+/−0.5%.

### EMBODIMENTS OF THE FIRST, SECOND, THIRD, FOURTH AND FIFTH ASPECTS OF THE INVENTION

### Embodiment E1

A pharmaceutical composition comprising the drug substance Compound 1, or the pharmaceutical composition according to any one of the first, second, third, fourth or fifth aspects of the invention, or any embodiments thereof, which comprises:
(i) talc; and
(ii) sodium stearyl fumarate.

### Embodiment E2

The pharmaceutical composition according to Embodiment E1 which comprises:
(i) between 0.1 and 1% w/w talc; and
(ii) between 0.5 and 3% w/w sodium stearyl fumarate.

### Embodiment E3

A pharmaceutical composition comprising the drug substance Compound 1, the pharmaceutical composition according to Embodiments E1 or E2, or the pharmaceutical composition according to any one of the first, second, third or fourth aspects of the invention, or any embodiments thereof, which comprises:
(i) starch or cellulose; and
(ii) hydroxypropyl cellulose or hydroxypropyl methylcellulose.

### Embodiment E4

The pharmaceutical composition according to Embodiment E3 which comprises:
(i) starch; and
(ii) hydroxypropyl cellulose.

### Embodiment E5

The pharmaceutical composition according to Embodiment E4 which comprises:
(i) between 5 and 25% w/w starch; and
(ii) between 1 and 5% w/w hydroxypropyl cellulose.

### Embodiment E6

The pharmaceutical composition according to Embodiment E4 which comprises:
(i) between 10 and 20% w/w starch; and
(ii) between 2 and 5% w/w hydroxypropyl cellulose.

### Embodiment E7

The pharmaceutical composition according to Embodiment E3 which comprises:
(i) between 30 and 70% w/w sugar alcohol;
(ii) between 5 and 25% w/w starch;
(iii) between 1 and 10% w/w low-substituted hydroxypropyl cellulose;
(iv) between 1 and 5% w/w hydroxypropyl cellulose;
(v) between 0.1 and 1% w/w talc; and
(vi) between 0.5 and 3% w/w sodium stearyl fumarate.

### Embodiment E8

The pharmaceutical composition according to Embodiment E3 which comprises:
(i) between 40 and 65% w/w sugar alcohol;
(ii) between 10 and 20% w/w starch;
(iii) between 2.5 and 7.5% w/w low-substituted hydroxypropyl cellulose;
(iv) between 2 and 4% w/w hydroxypropyl cellulose;
(v) between 0.25 and 0.75% w/w talc; and
(vi) between 0.5 and 2.5% w/w sodium stearyl fumarate.

### Embodiment E9

The pharmaceutical composition according to any one of Embodiments E3 to E8, wherein the starch is a partially pregelatinised maize starch.

### Embodiment E10

The pharmaceutical composition according any one of Embodiments E3 to E8, wherein the hydroxypropyl cellulose is high viscosity hydroxypropyl cellulose.

### Embodiment E11

The pharmaceutical composition according to Embodiment E3 which comprises:
(i) cellulose; and
(ii) hydroxypropyl methylcellulose.

### Embodiment E12

The pharmaceutical composition according to Embodiment E11 which comprises:
(i) between 10 and 60% w/w cellulose; and
(ii) between 1 and 5% w/w hydroxypropyl methylcellulose.

### Embodiment E13

The pharmaceutical composition according to Embodiment E11 which comprises:
(i) between 20 and 50% w/w cellulose; and
(ii) between 2 and 4% w/w hydroxypropyl methylcellulose.

### Embodiment E14

The pharmaceutical composition according to Embodiment E3 which comprises:
(i) between 25 and 50% w/w sugar alcohol;
(ii) between 10 and 60% w/w cellulose;
(iii) between 1 and 10% w/w low-substituted hydroxypropyl cellulose;
(iv) between 1 and 5% w/w hydroxypropyl methylcellulose;
(v) between 0.1 and 1% w/w talc; and
(vi) between 0.5 and 3% w/w sodium stearyl fumarate.

### Embodiment E15

The pharmaceutical composition according to Embodiment E3 which comprises:
(i) between 30 and 50% w/w sugar alcohol;
(ii) between 20 and 50% w/w cellulose;
(iii) between 2 and 8% w/w low-substituted hydroxypropyl cellulose;
(iv) between 1.5 and 5% w/w hydroxypropyl methylcellulose;
(v) between 0.25 and 0.75% w/w talc; and
(vi) between 0.5 and 2.5% w/w sodium stearyl fumarate.

### Embodiment E16

The pharmaceutical composition according to Embodiment E3 which comprises:
(i) between 35 and 50% w/w sugar alcohol;
(ii) between 30 and 45% w/w cellulose;
(iii) between 2.5 and 7.5% w/w low-substituted hydroxypropyl cellulose;
(iv) between 2 and 4% w/w hydroxypropyl methylcellulose;
(v) between 0.25 and 0.75% w/w talc; and
(vi) between 0.5 and 2.5% w/w sodium stearyl fumarate.

### Embodiment E17

The pharmaceutical composition according to Embodiment E3 which comprises:
(i) between 40 and 45% w/w sugar alcohol;
(ii) between 36 and 43% w/w cellulose;
(iii) between 3 and 7% w/w low-substituted hydroxypropyl cellulose;
(iv) between 2 and 4% w/w hydroxypropyl methylcellulose;
(v) between 0.25 and 0.75% w/w talc; and
(vi) between 1 and 2% w/w sodium stearyl fumarate.

### Embodiment E18

The pharmaceutical composition according to Embodiment E3 which comprises:
(i) 43% (+/−1%) w/w sugar alcohol;
(ii) 39% (+/−1%) w/w cellulose;
(iii) 5% (+/−0.5%) w/w low-substituted hydroxypropyl cellulose;
(iv) 3% (+/−0.5%) w/w hydroxypropyl methylcellulose;
(v) 0.5% (+/−0.2%) w/w talc; and
(vi) 1.5% (+/−0.25%) w/w sodium stearyl fumarate.

### Embodiment E19

The pharmaceutical composition according to Embodiment E3 which comprises:
(i) between 35 and 45% w/w sugar alcohol;
(ii) between 25 and 35% w/w cellulose;
(iii) between 2 and 8% w/w low-substituted hydroxypropyl cellulose;
(iv) between 2 and 4% w/w hydroxypropyl methylcellulose;
(v) between 0.25 and 0.75% w/w talc; and
(vi) between 0.5 and 2.5% w/w sodium stearyl fumarate.

### Embodiment E20

The pharmaceutical composition according to Embodiment E3 which comprises:
(i) between 37.5 and 42.5% w/w sugar alcohol;
(ii) between 27.5 and 32.5% w/w cellulose;
(iii) between 3 and 7% w/w low-substituted hydroxypropyl cellulose;
(iv) between 2 and 4% w/w hydroxypropyl methylcellulose;
(v) between 0.25 and 0.75% w/w talc; and
(vi) between 1 and 2% w/w sodium stearyl fumarate.

### Embodiment E21

The pharmaceutical composition according to Embodiment E3 which comprises:
(i) 39% (+/−1%) w/w sugar alcohol;
(ii) 30% (+/−1%) w/w cellulose;
(iii) 5% (+/−0.5%) w/w low-substituted hydroxypropyl cellulose;
(iv) 3% (+/−0.5%) w/w hydroxypropyl methylcellulose;
(v) 0.5% (+/−0.2%) w/w talc; and
(vi) 1.5% (+/−0.25%) w/w sodium stearyl fumarate.

### Embodiment E22

The pharmaceutical composition according to any one of Embodiments E11 to E21, wherein the cellulose is microcrystalline cellulose.

### Embodiment E23

The pharmaceutical composition according to any one of Embodiments E11 to E21, wherein the hydroxypropyl methylcellulose is 603 grade hydroxypropyl methylcellulose.

### Embodiment E24

The pharmaceutical composition according to any one of the first, second, third or fourth aspects of the invention and any one of Embodiments E1 to E6 or E11 to E13, which further comprises a sugar alcohol.

### Embodiment E25

The pharmaceutical composition according to Embodiment E24 wherein the pharmaceutical composition comprises at least 10, 15, 20, 25, or 30% w/w sugar alcohol.

### Embodiment E26

The pharmaceutical composition according to Embodiment E24 wherein the pharmaceutical composition comprises at least 30% w/w sugar alcohol.

### Embodiment E27

The pharmaceutical composition according to Embodiment E25 or E26 wherein the pharmaceutical composition comprises less than 45, 50, 55, 60, 65, 70 or 75% w/w sugar alcohol.

### Embodiment E28

The pharmaceutical composition according to Embodiment E27 wherein the pharmaceutical composition comprises less than 50% w/w sugar alcohol.

### Embodiment E29

The pharmaceutical composition according to any one of Embodiments E7, E8, E14 to E21, or E24 to E28 wherein the sugar alcohol has the general formula HOCH₂(CHOH)₄CH₂OH.

### Embodiment E30

The pharmaceutical composition according to any one of Embodiments E7, E8, E14 to E21, or E24 to E28 wherein the sugar alcohol is selected from xylitol, mannitol, and sorbitol.

### Embodiment E31

The pharmaceutical composition according to Embodiment E30 wherein the sugar alcohol is mannitol.

### Embodiment E32

The pharmaceutical composition according to any one of the first, second, third or fourth aspects of the invention and any one of Embodiments E1 to E31 wherein the pharmaceutical composition comprises 1 to 100 mg of drug substance.

### Embodiment E33

The pharmaceutical composition according to any one of the first, second, third or fourth aspects of the invention and any one of Embodiments E1 to E31 wherein the pharmaceutical composition comprises 1 to 75 mg of drug substance.

### Embodiment E34

The pharmaceutical composition according to any one of the first, second, third or fourth aspects of the invention and any one of Embodiments E1 to E31 wherein the pharmaceutical composition comprises 1, 10, 15, 25, 50 or 75 mg of drug substance.

### Embodiment E35

The pharmaceutical composition according to any one of the first, second, third or fourth aspects of the invention and any one of Embodiments E1 to E31 wherein the pharmaceutical composition comprises 15 mg of drug substance.

### Embodiment E36

The pharmaceutical composition according to any one of the first, second, third or fourth aspects of the invention and any one of Embodiments E1 to E31 wherein the pharmaceutical composition comprises 50 mg of drug substance.

### Embodiment E37

The pharmaceutical composition according to any one of the first, second, third or fourth aspects of the invention and any one of Embodiments E1 to E36 wherein the pharmaceutical composition comprises a gelatin capsule.

### Embodiment E38

The pharmaceutical composition according to any one of the first, second, third or fourth aspects of the invention and any one of Embodiments E1 to E37 wherein the drug substance Compound 1 is in free form.

### Embodiment E39

The pharmaceutical composition according to Embodiment E38 wherein the drug substance Compound 1 is in crystalline Form A.

### Embodiment E40

The pharmaceutical composition according to Embodiment E39 wherein crystalline Form A has an X-ray powder diffraction pattern with at least three peaks having angle of refraction 2 theta (θ) values selected from 10.7, 14.8, 18.7, 19.5 and 21.4° when measured using CuKα radiation, wherein said values are plus or minus 0.2° 2θ.

### Embodiment E41

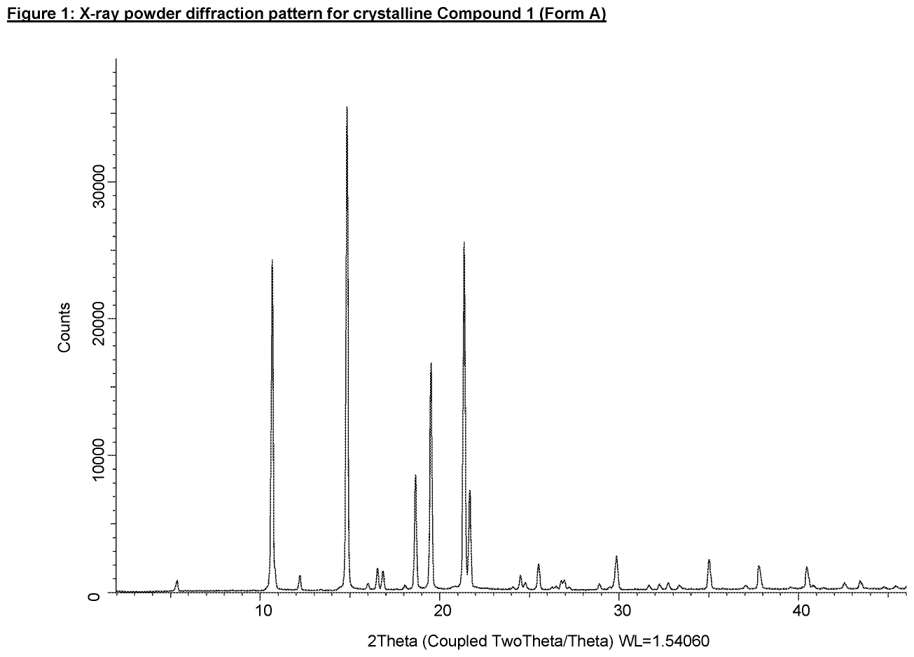

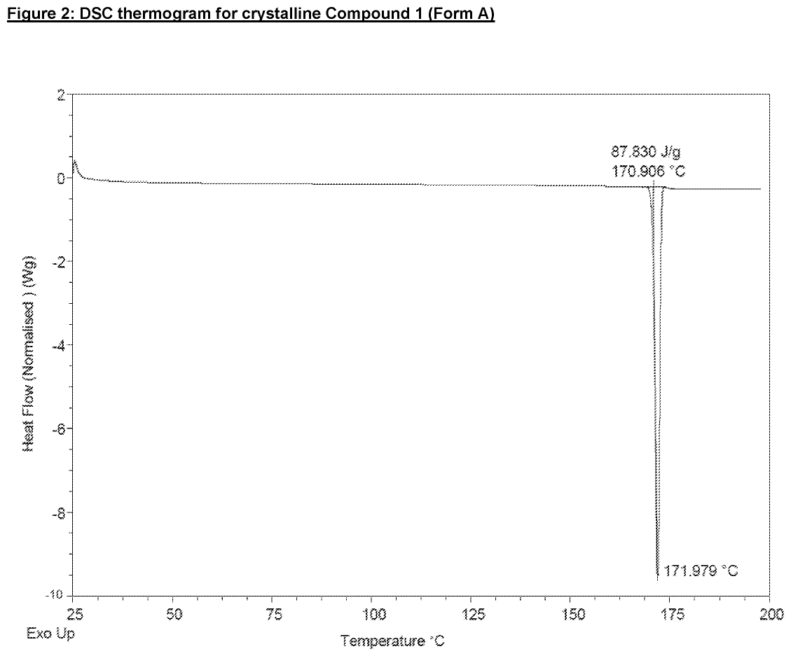

The pharmaceutical composition according to Embodiment E39 wherein crystalline Form A has an X-ray powder diffraction pattern substantially the same as that shown in FIG. 1 when measured using CuKα radiation.

### Embodiment E42

The pharmaceutical composition according to any one of Embodiments E1 to E41, wherein the pharmaceutical composition does not comprise a surfactant.

### Embodiment E43

A pharmaceutical composition comprising the drug substance Compound 1, or the pharmaceutical composition according to any one of the first, second, third, or fourth aspects of the invention, or any embodiments thereof, which further comprises a sugar alcohol.

### Embodiment E44

A pharmaceutical composition comprising the drug substance Compound 1, or the pharmaceutical composition according to any one of the first, second, third, or fourth aspects of the invention, or any embodiments thereof, which further comprises:
(i) a sugar alcohol; and
(ii) at least one further excipient selected from a filler, desintegrant, binder, glidant, and lubricant.

### Embodiment E45

A pharmaceutical composition comprising the drug substance Compound 1, or the pharmaceutical composition according to any one of the first, second, third, or fourth aspects of the invention, or any embodiments thereof, which further comprises:
(i) a sugar alcohol; and
(ii) at least two further excipients selected from a filler, desintegrant, binder, glidant, and lubricant.

### Embodiment E46

A pharmaceutical composition comprising the drug substance Compound 1, or the pharmaceutical composition according to any one of the first, second, third, or fourth aspects of the invention, or any embodiments thereof, which further comprises:
(i) a sugar alcohol; and
(ii) at least three further excipients selected from a filler, desintegrant, binder, glidant, and lubricant.

### Embodiment E47

A pharmaceutical composition comprising the drug substance Compound 1, or the pharmaceutical composition according to any one of the first, second, third, or fourth aspects of the invention, or any embodiments thereof, which further comprises:
(i) a sugar alcohol; and
(ii) at least four further excipients selected from a filler, desintegrant, binder, glidant, and lubricant.

### Embodiment E48

A pharmaceutical composition comprising the drug substance Compound 1, or the pharmaceutical composition according to any one of the first, second, third, or fourth aspects of the invention, or any embodiments thereof, which further comprises:
(i) a sugar alcohol
(ii) a filler;
(iii) a desintegrant;
(iv) a binder;
(v) a glidant; and
(vi) a lubricant.

### Embodiment E49

The pharmaceutical composition according to Embodiments E43 to E48, which comprises:
(i) between 30 and 70% w/w sugar alcohol;
(ii) between 5 and 60% w/w filler;
(iii) between 1 and 10% w/w desintegrant;
(iv) between 1 and 5% w/w binder;
(v) between 0.1 and 1% w/w glidant; and
(vi) between 0.5 and 3% w/w lubricant.

### Embodiment E50

The pharmaceutical composition according to Embodiments E43 to E48, which comprises:
(i) between 30 and 70% w/w sugar alcohol;
(ii) between 5 and 25% w/w filler;
(iii) between 1 and 10% w/w desintegrant;
(iv) between 1 and 5% w/w binder;
(v) between 0.1 and 1% w/w glidant; and
(vi) between 0.5 and 3% w/w lubricant.

### Embodiment E51

The pharmaceutical composition according to Embodiments E43 to E48, which comprises:
(i) between 30 and 70% w/w sugar alcohol;
(ii) between 5 and 25% w/w filler;
(iii) between 2.5 and 7.5% w/w desintegrant;
(iv) between 2 and 4% w/w binder;
(v) between 0.25 and 0.75% w/w glidant; and
(vi) between 0.5 and 2.5% w/w lubricant.

### Embodiment E52

The pharmaceutical composition according to Embodiments E48 to E51, wherein the ratio of % w/w sugar alcohol to % w/w filler is between 3.0 and 3.5.

### Embodiment E53

The pharmaceutical composition according to Embodiments E43 to E48, which comprises:
(i) between 25 and 50% w/w sugar alcohol;
(ii) between 10 and 60% w/w filler;
(iii) between 1 and 10% w/w desintegrant;
(iv) between 1 and 5% w/w binder;
(v) between 0.1 and 1% w/w glidant; and
(vi) between 0.5 and 3% w/w lubricant.

### Embodiment E54

The pharmaceutical composition according to Embodiments E43 to E48, which comprises:
(i) between 30 and 50% w/w sugar alcohol;
(ii) between 20 and 50% w/w filler;
(iii) between 2 and 8% w/w disintegrant;
(iv) between 1.5 and 5% w/w binder;
(v) between 0.25 and 0.75% w/w glidant; and
(vi) between 0.5 and 2.5% w/w lubricant.

### Embodiment E55

The pharmaceutical composition according to Embodiments E43 to E48, which comprises:
(i) between 35 and 50% w/w sugar alcohol;
(ii) between 30 and 45% w/w filler;
(iii) between 2.5 and 7.5% w/w disintegrant;
(iv) between 2 and 4% w/w binder;
(v) between 0.25 and 0.75% w/w glidant; and
(vi) between 0.5 and 2.5% w/w lubricant.

### Embodiment E56

The pharmaceutical composition according to Embodiments E43 to E48, which comprises:
(i) between 40 and 45% w/w sugar alcohol;
(ii) between 36 and 43% w/w filler;
(iii) between 3 and 7% w/w disintegrant;
(iv) between 2 and 4% w/w binder;
(v) between 0.25 and 0.75% w/w glidant; and
(vi) between 1 and 2% w/w lubricant.

### Embodiment E57

The pharmaceutical composition according to Embodiments E43 to E48, which comprises:
(i) 43% (+/−1%) w/w sugar alcohol;
(ii) 39% (+/−1%) w/w filler;
(iii) 5% (+/−0.5%) w/w disintegrant;
(iv) 3% (+/−0.5%) w/w binder;
(v) 0.5% (+/−0.2%) w/w glidant; and
(vi) 1.5% (+/−0.25%) w/w lubricant.

### Embodiment E58

The pharmaceutical composition according to Embodiments E43 to E48, which comprises:
(i) between 35 and 45% w/w sugar alcohol;
(ii) between 25 and 35% w/w filler;
(iii) between 2 and 8% w/w disintegrant;
(iv) between 2 and 4% w/w binder;
(v) between 0.25 and 0.75% w/w glidant; and
(vi) between 0.5 and 2.5% w/w lubricant.

### Embodiment E59

The pharmaceutical composition according to Embodiments E43 to E48, which comprises:
(i) between 37.5 and 42.5% w/w sugar alcohol;
(ii) between 27.5 and 32.5% w/w filler;
(iii) between 3 and 7% w/w disintegrant;
(iv) between 2 and 4% w/w binder;
(v) between 0.25 and 0.75% w/w glidant; and
(vi) between 1 and 2% w/w lubricant.

### Embodiment E60

The pharmaceutical composition according to Embodiments E43 to E48, which comprises:
(i) 39% (+/−1%) w/w sugar alcohol;
(ii) 30% (+/−1%) w/w filler;
(iii) 5% (+/−0.5%) w/w disintegrant;
(iv) 3% (+/−0.5%) w/w binder;
(v) 0.5% (+/−0.2%) w/w glidant; and
(vi) 1.5% (+/−0.25%) w/w lubricant.

### Embodiment E61

The pharmaceutical composition according to Embodiments E48, E49, and E53 to E60, wherein ratio of % w/w sugar alcohol to % w/w filler is less than 3.0.

### Embodiment E62

The pharmaceutical composition according to Embodiments E48, E49, and E53 to E60, wherein ratio of % w/w sugar alcohol to % w/w filler is between 1.0 and 3.0.

### Embodiment E63

The pharmaceutical composition according to Embodiments E48, E49, and E53 to E60, wherein ratio of % w/w sugar alcohol to % w/w filler is between 1.0 and 1.5.

### Embodiment E64

The pharmaceutical composition according to Embodiments E48, E49, and E53 to E60, wherein ratio of % w/w sugar alcohol to % w/w filler is 1.1 or 1.3.

### Embodiment E65

The pharmaceutical composition according to Embodiments E43 to E64, wherein the sugar alcohol has the general formula HOCH₂(CHOH)ₙCH₂OH wherein n is 2, 3 or 4.

### Embodiment E66

The pharmaceutical composition according to Embodiments E43 to E64, wherein the sugar alcohol has the general formula HOCH₂(CHOH)ₙCH₂OH wherein n is 3 or 4.

### Embodiment E67

The pharmaceutical composition according to Embodiments E43 to E64, wherein the sugar alcohol has the general formula HOCH₂(CHOH)₄CH₂OH.

### Embodiment E68

The pharmaceutical composition according to Embodiments E43 to E64, wherein the sugar alcohol is selected from erythritol, xylitol, mannitol, sorbitol, isomalt, maltitol and lactitol.

### Embodiment E69

The pharmaceutical composition according to Embodiments E43 to E64, wherein the sugar alcohol is selected from xylitol, mannitol, and sorbitol.

### Embodiment E70

The pharmaceutical composition according to Embodiments E43 to E64, wherein the sugar alcohol is mannitol.

### Embodiment E71

The pharmaceutical composition according to Embodiments E44 to E70, wherein the disintegrant is low-substituted hydroxypropyl cellulose.

### Embodiment E72

The pharmaceutical composition according to Embodiments E44 to E71, wherein the glidant is talc.

### Embodiment E73

The pharmaceutical composition according to Embodiments E44 to E72, wherein the lubricant sodium stearyl fumarate.

### Embodiment E74

The pharmaceutical composition according to Embodiments E50 to E52, wherein the filler is starch.

### Embodiment E75

The pharmaceutical composition according to Embodiments E53 to E64, wherein the filler is microcrystalline cellulose.

### Embodiment E76

The pharmaceutical composition according to Embodiments E50 to E52, wherein the binder is hydroxypropyl cellulose.

### Embodiment E77

The pharmaceutical composition according to Embodiments E53 to E64, wherein the binder is hydroxypropyl methylcellulose.

### Embodiment E78

The pharmaceutical composition according to any one of the first, second, third or fourth aspects of the invention and any one of Embodiments E43 to E77 wherein the pharmaceutical composition comprises 1 to 100 mg of drug substance.

### Embodiment E79

The pharmaceutical composition according to any one of the first, second, third or fourth aspects of the invention and any one of Embodiments E43 to E77 wherein the pharmaceutical composition comprises 1 to 75 mg of drug substance.

### Embodiment E80

The pharmaceutical composition according to any one of the first, second, third or fourth aspects of the invention and any one of Embodiments E43 to E77 wherein the pharmaceutical composition comprises 1, 10, 15, 25, 50 or 75 mg of drug substance.

### Embodiment E81

The pharmaceutical composition according to any one of the first, second, third or fourth aspects of the invention and any one of Embodiments E43 to E77 wherein the pharmaceutical composition comprises 15 mg of drug substance.

### Embodiment E82

The pharmaceutical composition according to any one of the first, second, third or fourth aspects of the invention and any one of Embodiments E43 to E77 wherein the pharmaceutical composition comprises 50 mg of drug substance.

### Embodiment E83

The pharmaceutical composition according to any one of the first, second, third or fourth aspects of the invention and any one of Embodiments E43 to E82 wherein the pharmaceutical composition comprises a gelatin capsule.

### Embodiment E84

The pharmaceutical composition according to any one of the first, second, third or fourth aspects of the invention and any one of Embodiments E43 to E83 wherein the drug substance Compound 1 is in free form.

### Embodiment E85

The pharmaceutical composition according to Embodiment E84 wherein the drug substance Compound 1 is in crystalline Form A.

### Embodiment E86

The pharmaceutical composition according to Embodiment E85 wherein crystalline Form A has an X-ray powder diffraction pattern with at least three peaks having angle of refraction 2 theta (θ) values selected from 10.7, 14.8, 18.7, 19.5 and 21.4° when measured using CuKα radiation, wherein said values are plus or minus 0.2° 2θ.

### Embodiment E87

The pharmaceutical composition according to Embodiment E85 wherein crystalline Form A has an X-ray powder diffraction pattern substantially the same as that shown in FIG. 1 when measured using CuKα radiation.

### Embodiment E88

The pharmaceutical composition according to any one of Embodiments E43 to E87, wherein the pharmaceutical composition does not comprise a surfactant.

In the fifth aspect of the invention as described hereinabove, the term “comprising” or “comprises” may be substituted with “consisting essentially of,” “consists essentially of,” “consisting of,” or “consists of.”

### EMBODIMENTS OF THE SIXTH ASPECT OF THE INVENTION

### Embodiment F1

A pharmaceutical composition according to any one of the first, second, third, fourth or fifth aspect of the invention, or any embodiments thereof, for use in the treatment or prevention of Alzheimer's disease.

### Embodiment F2

The pharmaceutical composition for the use according to Embodiment F1, wherein the drug substance Compound 1 is used at a dose of between 10 and 30 mg per day.

### Embodiment F3

The pharmaceutical composition for the use according to Embodiment F1, wherein the drug substance Compound 1 is used at a dose of between 30 and 100 mg per day.

### Embodiment F4

The pharmaceutical composition for the use according to Embodiment F1, wherein the drug substance Compound 1 is used at a dose of between 30 and 50 mg per day.

### Embodiment F5

The pharmaceutical composition for the use according to Embodiment F1, wherein the drug substance Compound 1 is used at a dose of 15 mg per day.

### Embodiment F6

The pharmaceutical composition for the use according to Embodiment F1, wherein the drug substance Compound 1 is used at a dose of 50 mg per day.

### EMBODIMENTS OF THE SEVENTH ASPECT OF THE INVENTION

### Embodiment G1

A method for the treatment or prevention of Alzheimer's disease which method comprises administering to a patient in need thereof the pharmaceutical composition according to any one of the first, second, third, fourth or fifth aspect of the invention, or any embodiments thereof, comprising a therapeutically effective amount of drug substance Compound 1.

### Embodiment G2

The method according to Embodiment G1, wherein the drug substance Compound 1 is used at a dose of between 10 and 30 mg per day.

### Embodiment G3

The method according to Embodiment G1, wherein the drug substance Compound 1 is used at a dose of between 30 and 100 mg per day.

### Embodiment G4

The method according to Embodiment G1, wherein the drug substance Compound 1 is used at a dose of between 30 and 50 mg per day.

### Embodiment G5

The method according to Embodiment G1, wherein the drug substance Compound 1 is used at a dose of 15 mg per day.

### Embodiment G6

The method according to Embodiment G1, wherein the drug substance Compound 1 is used at a dose of 50 mg per day.

### EMBODIMENTS OF THE EIGHTH ASPECT OF THE INVENTION

### Embodiment H1

Use of a pharmaceutical composition according to any one of the first, second, third, fourth or fifth aspect of the invention, or any embodiments thereof, for the treatment or prevention of Alzheimer's disease.

### Embodiment H2

The use according to Embodiment H1, wherein the drug substance Compound 1 is used at a dose of between 10 and 30 mg per day.

### Embodiment H3

The use according to Embodiment H1, wherein the drug substance Compound 1 is used at a dose of between 30 and 100 mg per day.

### Embodiment H4

The use according to Embodiment H1, wherein the drug substance Compound 1 is used at a dose of between 30 and 50 mg per day.

### Embodiment H5

The use according to Embodiment H1, wherein the drug substance Compound 1 is used at a dose of 15 mg per day.

### Embodiment H6

The use according to Embodiment H1, wherein the drug substance Compound 1 is used at a dose of 50 mg per day.

### EMBODIMENTS OF THE NINTH ASPECT OF THE INVENTION

### Embodiment I1

Use of the drug substance Compound 1 for the manufacture of a pharmaceutical composition according to any one of the first, second, third, fourth or fifth aspect of the invention, or any embodiments thereof, for the treatment or prevention of Alzheimer's disease.

### Embodiment I2

The use according to Embodiment I1, wherein the drug substance Compound 1 is used for the treatment or prevention of Alzheimer's disease at a dose of between 10 and 30 mg per day.

### Embodiment I3

The use according to Embodiment I1, wherein the drug substance Compound 1 is used for the treatment or prevention of Alzheimer's disease at a dose of between 30 and 100 mg per day.

### Embodiment I4

The use according to Embodiment I1, wherein the drug substance Compound 1 is used for the treatment or prevention of Alzheimer's disease at a dose of between 30 and 50 mg per day.

### Embodiment I5

The use according to Embodiment I1, wherein the drug substance Compound 1 is used for the treatment or prevention of Alzheimer's disease at a dose of 15 mg per day.

### Embodiment I6

The use according to Embodiment I1, wherein the drug substance Compound 1 is used for the treatment or prevention of Alzheimer's disease at a dose of 50 mg per day.

### EMBODIMENTS OF THE TENTH ASPECT OF THE INVENTION

### Embodiment J1

A process for the preparation of a pharmaceutical composition comprising the drug substance Compound 1 wherein the drug substance is co-milled with a sugar alcohol.

### Embodiment J2

The process according to Embodiment J1 wherein the sugar alcohol has the general formula HOCH₂(CHOH)ₙCH₂OH wherein n is 2, 3 or 4.

### Embodiment J3

The process according to Embodiment J1 wherein the sugar alcohol has the general formula HOCH₂(CHOH)ₙCH₂OH wherein n is 3 or 4.

### Embodiment J4

The process according to Embodiment J1 wherein the sugar alcohol has the general formula HOCH₂(CHOH)₄CH₂OH.

### Embodiment J5

The process according to Embodiment J1 wherein the sugar alcohol is selected from erythritol, xylitol, mannitol, sorbitol, isomalt, maltitol and lactitol.

### Embodiment J6

The process according to Embodiment J1 wherein the sugar alcohol is selected from xylitol, mannitol, and sorbitol.

### Embodiment J7

The process according to Embodiment J1 wherein the sugar alcohol is mannitol.

### Embodiment J8

The process according to any one of Embodiments J1 to J7 wherein the drug substance Compound 1 is co-milled with at least 20, 25, 30, 35, 40, or 45% w/w sugar alcohol.

### Embodiment J9

The process according to any one of Embodiments J1 to J7 wherein the drug substance Compound 1 is co-milled with at least 30% w/w sugar alcohol.

### Embodiment J10

The process according to any one of Embodiments J1 to J9 wherein the drug substance Compound 1 is co-milled with less than 55, 60, 65, 70, or 80% w/w sugar alcohol.

### Embodiment J11

The process according to any one of Embodiments J1 to J9 wherein the drug substance Compound 1 is co-milled with less than 55% w/w sugar alcohol.

### Embodiment J12

The process according to any one of Embodiments J1 to J7 wherein 50% w/w drug substance Compound 1 is co-milled with 50% w/w sugar alcohol.

### Embodiment J13

The pharmaceutical composition according to any one of the first, second, third, fourth or fifth aspect of the invention, or any embodiments thereof, wherein, during the preparation thereof, the drug substance Compound 1 is co-milled with a sugar alcohol.

### Embodiment J14

The pharmaceutical composition according to Embodiment J13 wherein the sugar alcohol has the general formula HOCH₂(CHOH)ₙCH₂OH wherein n is 2, 3 or 4.

### Embodiment J15

The pharmaceutical composition according to Embodiment J13 wherein the sugar alcohol has the general formula HOCH₂(CHOH)ₙCH₂OH wherein n is 3 or 4.

### Embodiment J16

The pharmaceutical composition according to Embodiment J13 wherein the sugar alcohol has the general formula HOCH₂(CHOH)₄CH₂OH.

### Embodiment J17

The pharmaceutical composition according to Embodiment J13 wherein the sugar alcohol is selected from erythritol, xylitol, mannitol, sorbitol, isomalt, maltitol and lactitol.

### Embodiment J18

The pharmaceutical composition according to Embodiment J13 wherein the sugar alcohol is selected from xylitol, mannitol, and sorbitol.

### Embodiment J19

The pharmaceutical composition according to Embodiment J13 wherein the sugar alcohol is mannitol.

### Embodiment J20

The pharmaceutical composition according to any one of Embodiments J13 to J19 wherein the drug substance Compound 1 is co-milled with at least 20, 25, 30, 35, 40, or 45% w/w sugar alcohol.

### Embodiment J21

The pharmaceutical composition according to any one of Embodiments J13 to J19 wherein the drug substance Compound 1 is co-milled with at least 30% w/w sugar alcohol.

### Embodiment J22

The pharmaceutical composition according to any one of Embodiments J13 to J21 wherein the drug substance Compound 1 is co-milled with less than 55, 60, 65, 70, or 80% w/w sugar alcohol.

### Embodiment J23

The pharmaceutical composition according to any one of Embodiments J13 to J21 wherein the drug substance Compound 1 is co-milled with less than 55% w/w sugar alcohol.

### Embodiment J24

The pharmaceutical composition according to any one of Embodiments J13 to J19 wherein 50% w/w drug substance Compound 1 is co-milled with 50% w/w sugar alcohol.

### Definitions

As used herein, the terms “Compound 1”, “Cmpd 1” or “the drug substance Compound 1” refer to N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide and having the following structural formula:

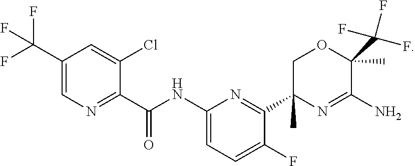 [SMILES: C[C@@]1(c2nc(NC(=O)c3ncc(C(F)(F)F)cc3Cl)ccc2F)CO[C@@](C)(C(F)(F)F)C(N)=N1]

In Example 1, using an alternative chemical naming format, “Compound 1” is also referred to as 3-chloro-5-trifluoromethyl-pyridine-2-carboxylic acid [6-((3R,6R)-5-amino-3,6-dimethyl-6-trifluoromethyl-3,6-dihydro-2H-[1,4]oxazin-3-yl)-5-fluoro-pyridin-2-yl]-amide.

The terms “Compound 1”, “Cmpd 1”, “the drug substance Compound 1” and its corresponding full chemical name are used interchangeably throughout the description of the invention. It is intended that the term refers to the compound in either free form, pharmaceutically acceptable salt form, crystalline form or co-crystal form, unless the context clearly indicates that only one form of the compound is intended. Compound 1 is described in WO 2012/095469 A1, Example 34. WO 2012/095469 A1 is incorporated herewith by reference in its entirety, in particular the disclosure related to the synthesis of Example 34.

As used herein the term “Cmax” refers to the maximum plasma concentration that the drug substance achieves following administration of a single dose. In the first aspect of the invention, the Cmax value of the drug substance measured in ng/mL is defined as a function of the drug substance dose in mg multiplied by a factor of 2.4; within a +/−range defined by the drug substance dose in mg multiplied by a factor of 0.7. For example, a pharmaceutical composition comprising 50 mg drug substance would fall within the scope of the invention if, subsequent to administration to a human subject, the plasma Cmax value fell within the range of 85 to 155 ng/ml. As a further example, a pharmaceutical composition comprising 15 mg drug substance would fall within the scope of the invention if, subsequent to administration to a human subject, the plasma Cmax value fell within the range of 25.5 to 46.5 ng/ml.

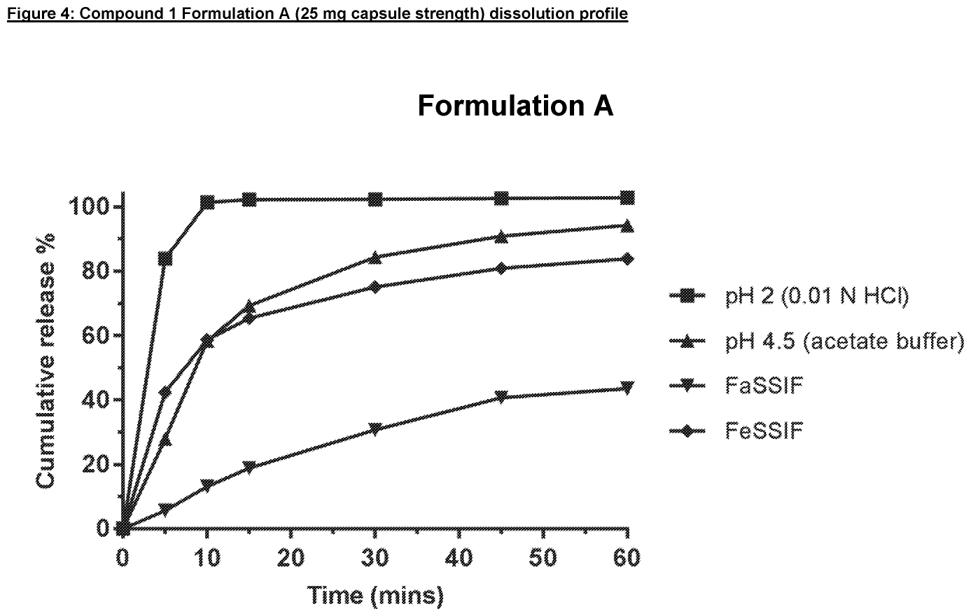

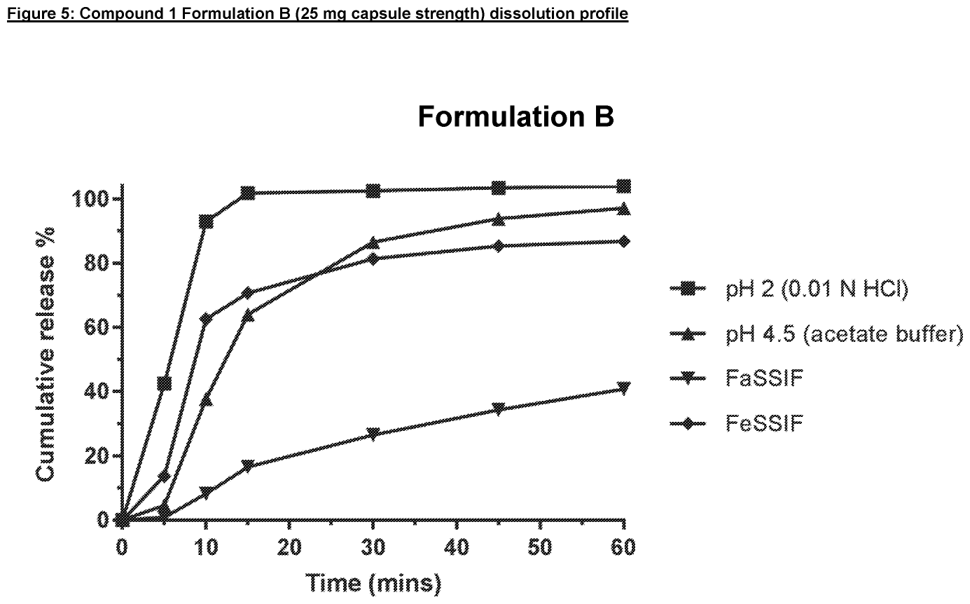

As used herein, the term “dissolution profile” refers to the rate and extent of drug substance release when a pharmaceutical composition of the present invention is dissolved in a test medium/buffer using the basket method described in US Pharmacopeia Chapter <711> “Dissolution”) edition 39-NF 34 and the following testing parameters—Dissolution medium: acetate buffer pH 4.5 (500 ml for dosage strengths up to 15 mg; 900 ml for dosage strengths above 15 mg); Apparatus 1: 100 rpm; Total Measurement Time: 60 minutes; and Temperature: 37±0.5° C. The dissolution profiles of pharmaceutical compositions comprising Compound 1 are shown in FIGS. 3 to 7 and a more detailed description of how the dissolution profiles are created is provided in Example 9 herein.

As used in the context of the third aspect of the invention, the term “blend” refers to the content of the pharmaceutical composition in unit dose solid form. In the context of a pharmaceutical composition which is a capsule, the “blend” refers to the fill content of said capsule.

As used herein, the term “as determined by mercury porosity” refers to the methodology set out in US Pharmacopeia Chapter <267> “Porosimetry by Mercury Intrusion” edition 39-NF 34. Further details are provided in Example 10 herein.

As used herein, the term “% w/w” refers to the percentage mass/mass. In the fourth aspect of the invention, the drug substance is present within the pharmaceutical composition in an amount greater than 7% w/w. It is intended that the % w/w value defined by the fourth aspect of the invention represents the percentage mass of the drug substance/capsule fill weight in the absence of the empty capsule shell weight. For example, a pharmaceutical composition comprising 15 mg drug substance, 180 mg capsule fill mix (or blend), and a capsule shell weighing 61 mg would have a % w/w value of 15/180=8.3%. As a further example, a pharmaceutical composition comprising 50 mg drug substance, 240 mg capsule fill mix (or blend), and a capsule shell weighing 61 mg would have a % w/w value of 50/240=20.8%.

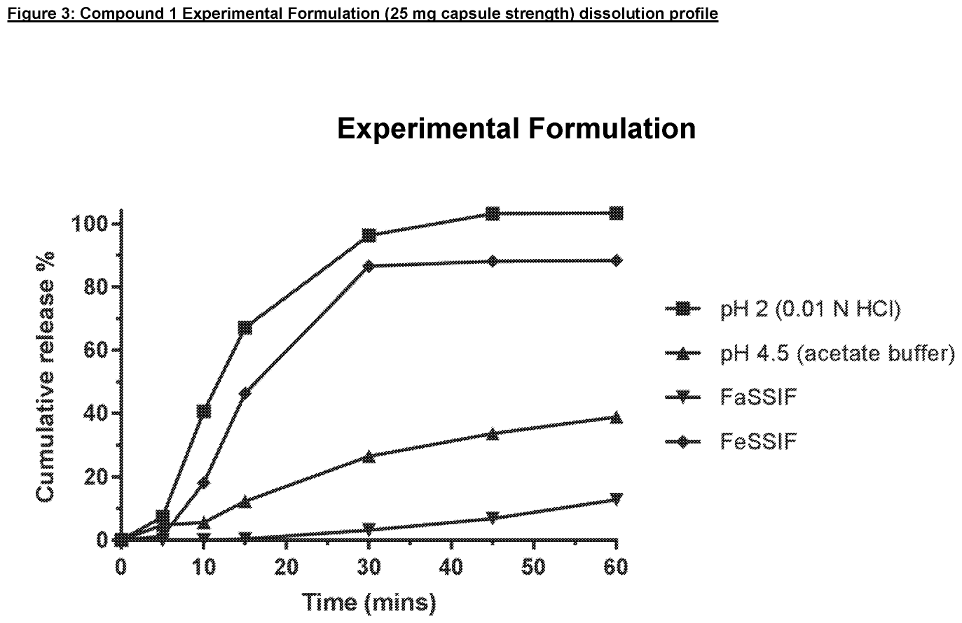

As used herein, the term “Form A” refers to a crystalline form of free base Compound 1 which has an X-ray powder diffraction pattern substantially the same as the X-ray powder diffraction pattern shown in FIG. 1 when measured using CuKα radiation. “Form A” may thus be defined as a crystalline form Compound 1 which has an X-ray powder diffraction pattern with at least one, two, three, four or five peaks having angle of refraction 2 theta (θ) values selected from 10.7, 14.8, 18.7, 19.5, 21.4, 21.7, 25.5, 29.9, 35.0 and 37.8° when measured using CuKα radiation, more particularly wherein said values are plus or minus 0.2° 2θ. “Form A” may also be defined as a crystalline form Compound 1 which has an X-ray powder diffraction pattern with at least one, two, three, four or five peaks having angle of refraction 2 theta (θ) values selected from 10.7, 14.8, 18.7, 19.5 and 21.4° when measured using CuKα radiation, more particularly wherein said values are plus or minus 0.2° 2θ. Additionally, “Form A” may be defined as a crystalline form Compound 1 which has an X-ray powder diffraction pattern with at least one, two or three peaks having angle of refraction 2 theta (θ) values selected from 10.7, 14.8 and 19.5° when measured using CuKα radiation, more particularly wherein said values are plus or minus 0.2° 2θ. “Form A” may also be defined as a crystalline form Compound 1 having an X-ray powder diffraction pattern substantially the same as that shown in shown FIG. 1 when measured using CuKα radiation. Additionally, “Form A” may be defined as a crystalline form of free base Compound 1 having an onset of melting at about 171° C. or a differential scanning calorimetry (DSC) thermogram substantially the same as that shown in shown in FIG. 2. For details see Example 4.

The term “substantially the same” with reference to X-ray diffraction peak positions means that typical peak position and intensity variability are taken into account. For example, one skilled in the art will appreciate that the peak positions (2Θ) will show some inter-apparatus variability, typically as much as 0.2°. Further, one skilled in the art will appreciate that relative peak intensities will show inter-apparatus variability as well as variability due to degree of crystallinity, preferred orientation, prepared sample surface, and other factors known to those skilled in the art, and should be taken as a qualitative measure only. One of ordinary skill in the art will also appreciate that an X-ray diffraction pattern may be obtained with a measurement error that is dependent upon the measurement conditions employed. In particular, it is generally known that intensities in an X-ray diffraction pattern may fluctuate depending upon measurement conditions employed. It should be further understood that relative intensities may also vary depending upon experimental conditions and, accordingly, the exact order of intensity should not be taken into account. Additionally, a measurement error of diffraction angle for a conventional X-ray diffraction pattern is typically about 5% or less, and such degree of measurement error should be taken into account as pertaining to the aforementioned diffraction angles. Consequently, it is to be understood that the crystal form of the instant invention is not limited to the crystal form that provides an X-ray diffraction pattern completely identical to the X-ray diffraction pattern depicted in the accompanying FIG. 1 disclosed herein. Any crystal forms that provide X-ray diffraction patterns substantially identical to that disclosed in the accompanying FIG. 1 fall within the scope of the present invention. The ability to ascertain substantial identities of X-ray diffraction patterns is within the purview of one of ordinary skill in the art. An expression referring to a crystalline form of Compound 1 having “an X-ray powder diffraction pattern substantially the same as the X-ray powder diffraction pattern shown in Figure X” may be interchanged with an expression referring to a crystalline form of Compound 1 having “an X-ray powder diffraction pattern characterised by the representative X-ray powder diffraction pattern shown in Figure X”.

As used herein, the term “Alzheimer's disease” or “AD” encompasses both preclinical and clinical Alzheimer's disease unless the context makes clear that either only preclinical Alzheimer's disease or only clinical Alzheimer's disease is intended.

As used herein, the term “treatment of Alzheimer's disease” refers to the administration of Compound 1 to a patient in order to ameliorate at least one of the symptoms of Alzheimer's disease.

As used herein, the term “prevention of Alzheimer's disease” refers to the prophylactic treatment of AD; or delaying the onset or progression of AD. For example, the onset or progression of AD is delayed for at least 0.5, 1, 2, 3, 4, 5, 6, 7, 8, 9, or 10 years. In one embodiment, “prevention of Alzheimer's disease” refers to the prophylactic treatment of preclinical AD; or delaying the onset or progression of preclinical AD. In a further embodiment, the onset or progression of preclinical AD is delayed for at least 0.5, 1, 2, 3, 4, 5, 6, 7, 8, 9, or 10 years. In another embodiment, “prevention of Alzheimer's disease” refers to the prophylactic treatment of clinical AD; or delaying the onset or progression of clinical AD. In a further embodiment, the onset or progression of clinical AD is delayed for at least 0.5, 1, 2, 3, 4, 5, 6, 7, 8, 9, or 10 years.

As used herein, the term “clinical Alzheimer's disease” or “clinical AD” encompasses both Mild Cognitive Impairment (MCI) due to AD and dementia due to AD, unless the context makes clear that either only MCI due to AD or dementia due to AD is intended. The European Medicines Agency (EMA) in its ‘Draft guidelines on the clinical investigation of medicines for the treatment of AD and other dementias’ (EMA/Committee for Medicinal Products for Human Use (CHMP)/539931/2014) summarises the National Institute on Aging criteria for the diagnosis of MCI due to AD and AD dementia as set out below.

Diagnosis of MCI due to AD requires evidence of intra-individual decline, manifested by:
a) A change in cognition from previously attained levels, as noted by self- or informant report and/or the judgment of a clinician.
b) Impaired cognition in at least one domain (but not necessarily episodic memory) relative to age- and education-matched normative values; impairment in more than one cognitive domain is permissible.
c) Preserved independence in functional abilities, although the criteria also accept ‘mild problems’ in performing instrumental activities of daily living (IADL) even when this is only with assistance (i.e. rather than insisting on independence, the criteria allow for mild dependence due to functional loss).
d) No dementia, which nominally is a function of c (above).
e) A clinical presentation consistent with the phenotype of AD in the absence of other potentially dementing disorders. Increased diagnostic confidence may be suggested by
   1) Optimal: A positive Aβ biomarker and a positive degeneration biomarker
   2) Less optimal:
      i. A positive Aβ biomarker without a degeneration biomarker
      ii. A positive degeneration biomarker without testing for Aβ biomarkers

Diagnosis of AD dementia requires:
a) The presence of dementia, as determined by intra-individual decline in cognition and function.
b) Insidious onset and progressive cognitive decline.
c) Impairment in two or more cognitive domains; although an amnestic presentation is most common, the criteria allow for diagnosis based on nonamnestic presentations (e.g. impairment in executive function and visuospatial abilities).
d) Absence of prominent features associated with other dementing disorders.

Increased diagnostic confidence may be suggested by the biomarker algorithm discussed in the MCI due to AD section above.

As used herein, the term “preclinical Alzheimer's disease” or “preclinical AD” refers to the presence of in vivo molecular biomarkers of AD in the absence of clinical symptoms. The National Institute on Aging and Alzheimer's Association provide a scheme, shown in Table 1 below, which sets out the different stages of preclinical AD (Sperling et al., 2011).

TABLE 1
Preclinical AD staging categories

| | | | Markers of | Evidence of |
|---|---|---|---|---|
| | | Aβ (PET | neuronal injury | subtle |
| Stage | Description | or CSF) | (tau, FDG, sMRI) | cognitive change |
| Stage 1 | Asymptomatic cerebral | Positive | Negative | Negative |
| | amyloidosis | | | |
| Stage 2 | Asymptomatic amyloidosis + | Positive | Positive | Negative |
| | “downstream” neurodegeneration | | | |
| Stage 3 | Amyloidosis + neuronal injury + | Positive | Positive | Positive |
| | subtle cognitive/behavioral decline | | | |

sMRI = structural magnetic resonance imaging

As used herein, the term “patient” refers to a human subject.

As used herein, the term “pharmaceutically acceptable salt” refers to salts that retain the biological effectiveness of Compound 1 and which typically are not biologically or otherwise undesirable (Stahl H, Wermuth C, 2011).

As used herein, a “pharmaceutical composition” comprises Compound 1 and at least one pharmaceutically acceptable carrier, in a unit dose solid form suitable for oral administration (typically a capsule, more particularly a hard gelatin capsule). A list of pharmaceutically acceptable carriers can be found in Remington's Pharmaceutical Sciences.

As used herein, the term “low-substituted hydroxypropyl cellulose” refers to a disintegrant with only a low level of hydroxypropoxy groups in the cellulose backbone, for example having an average number of hydroxypropoxy groups per glucose ring unit of the cellulose backbone of about 0.2. Low-substituted hydroxypropyl cellulose is not the same as hydroxypropyl cellulose which, for example, has an average number of hydroxypropoxy groups per glucose ring unit of the cellulose backbone of about 3.5.

As used herein, the terms “hydroxypropyl methycellulose” and “hypromellose” refer to cellulose, 2-hydroxypropyl methyl ether (CAS 9004-65-3), and are used interchangeably.

The term “a therapeutically effective amount” refers to an amount of Compound 1 that will elicit inhibition of BACE-1 in a patient as evidenced by a reduction in CSF or plasma Aβ 1-40 levels relative to an initial baseline value. Aβ 1-40 levels may be measured using standard immunoassay techniques, for example Meso Scale Discovery (MSD) 96-well MULTI-ARRAY human/rodent (4G8) Aβ40 Ultrasensitive Assay (#K110FTE-3, Meso Scale Discovery, Gaithersburg, USA).

As used herein, the term “sugar alcohol” refers to a compound having the following general formula HOCH₂(CHOH)ₙCH₂OH wherein n is 2, 3 or 4; or a compound of formula (I)

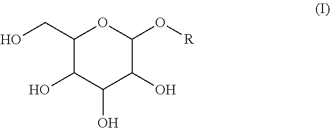

wherein R represents a pentahydroxyhexyl group which is attached to the rest of the molecule by a bond to any one of the carbon atoms within the pentahydroxyhexyl group. In a one embodiment, the term “sugar alcohol” refers to a compound derived from sugar having the following general formula HOCH₂(CHOH)ₙCH₂OH wherein n is 2, 3 or 4. In another embodiment, the term “sugar alcohol” refers to a compound derived from sugar having the following general formula HOCH₂(CHOH)ₙCH₂OH wherein n is 3 or 4. The expression “derived from sugar” is intended to mean that the chemical structure of the sugar alcohol is derived from sugar and not, necessarily, that the sugar alcohol material itself is derived from sugar. Examples of sugar alcohols include, but are not limited to, erythritol, xylitol, mannitol, sorbitol, isomalt, maltitol and lactitol. In yet another embodiment, the sugar alcohol is mannitol.

As used herein, the term “surfactant” refers to any pharmaceutically acceptable agent that is absorbed at phase interfaces and effectively lowers the surface tension between Compound 1 and aqueous fluids (Sinko P J, Martin A N, 2011).

As used herein, the term “filler” refers to a substance added to a pharmaceutical composition to increase the weight and/or the size of the pharmaceutical composition. Pharmaceutically acceptable fillers are described in Remington's Pharmaceutical Sciences and listed in Handbook of Pharmaceutical Excipients, Sheskey et al, 2017. In one embodiment the filler is starch (e.g., pregelatinized starch) or cellulose (e.g., microcrystalline cellulose). In another embodiment the filler is starch. In yet another embodiment the filler is microcrystalline cellulose.

As used herein, the term “disintegrant” refers to a substance added to a pharmaceutical composition to help it break apart (disintegrate), e.g., after administration, and release the active ingredient, such as the drug substance Compound 1. Pharmaceutically acceptable disintegrants are described in Remington's Pharmaceutical Sciences and listed in Handbook of Pharmaceutical Excipients, Sheskey et al, 2017. In one embodiment the disintegrant is low substituted hydroxypropyl cellulose.

As used herein, the term “binder” refers to a substance added to a pharmaceutical composition to help literally “bind together” the individual components of a pharmaceutical composition. Pharmaceutically acceptable binders are described in Remington's Pharmaceutical Sciences and listed in Handbook of Pharmaceutical Excipients, Sheskey et al, 2017. In one embodiment the binder is hydroxypropyl cellulose or hydroxypropyl methyl cellulose. In another embodiment the binder is hydroxypropyl cellulose. In yet another embodiment the binder is hydroxypropyl methyl cellulose.

As used herein, the term “glidant” refers to a substance added to a pharmaceutical composition to enhance the flow of a mixture, e.g., a granular mixture, by, e.g., reducing interparticle friction. Pharmaceutically acceptable glidants are described in Remington's Pharmaceutical Sciences and listed in Handbook of Pharmaceutical Excipients, Sheskey et al, 2017. In one embodiment the glidant is talc.

As used herein, the term “lubricant” refers to a substance added to a dosage form to help reduce the adherence of a granule or powder to equipment surfaces. Pharmaceutically acceptable lubricants are described in Remington's Pharmaceutical Sciences and listed in Handbook of Pharmaceutical Excipients, Sheskey et al, 2017. In one embodiment the lubricant is sodium stearyl fumarate.

### List of Abbreviations

| Abbreviation | Description |
|---|---|
| ACN | acetonitrile |
| APP | amyloid precursor protein |
| Aβ | beta-amyloid peptide |
| aq. | aqueous |
| AUClast | The area under the plasma concentration-time curve from time zero to the |
| | time of the last quantifiable concentration, calculated using the linear |
| | trapezoidal rule [mass × time/volume] |
| AUCinf | The area under the plasma concentration-time curve from time zero to |
| | infinity, calculated using the linear trapezoidal rule calculated as AUCinf = |
| | AUClast + Clast/Lambda_z, where Clast is the last measurable |
| | oncentration and Lambda_z is the elimination rate constant [mass × |
| | time/volume] |
| Aβ40 | beta-amyloid peptide 40 |
| BACE-1 | beta site APP cleaving enzyme-1 |
| BACE-2 | beta site APP cleaving enzyme -2 |
| BACE | beta site APP cleaving enzyme |
| Boc₂O | di-tert-butyl dicarbonate |
| BuLi or nBuLi | n-butyllithium |
| C | concentration |
| CI | confidence interval |
| conc. | concentrated |
| Cpd | compound |
| CSF | cerebrospinal fluid |
| d | day |
| DCM | dichloromethane |
| DDI | drug-drug interaction |
| DEA | diethylamine |
| DMAP | 4-dimethylaminopyridine |
| DMF | N,N-dimethylformamide |
| DMSO | dimethylsulfoxide |
| DS | drug substance |
| DSC | differential scanning calorimetry |
| EDC | 1-(3-dimethylaminopropyl)-3-ethylcarbodiimide hydrochloride |
| EDTA | ethylenediamine tetraethyl acetate |
| EF | experimental formulation |
| ESI | electrospray ionisation |
| EtOAc | ethyl acetate |
| FA | Formulation A |
| FB | Formulation B |
| FaSSIF | fasted state simulated intestinal fluid |
| FeSSIF | fed state simulated intestinal fluid |
| g | gram/gravitational acceleration |
| h, hr | hour(s) |
| HDPE | high density polyethylene |
| HGC | hard gelatin capsule |
| HOAt | 1-hydroxy-7-azabenzotriazole |
| HPLC, LC | high-performance liquid chromatography, liquid chromatography |
| IC₅₀ | inhibitory concentration 50 |
| IPAc | isopropyl acetate |
| K₃EDTA | tri-potassium ethylenediaminetetraacetic acid |
| kg | kilogram |
| LC-MS/MS | tandem mass spectrometry |
| LLOQ | lower limit of quantification |
| MeOH | methanol |
| m | meter |
| min | minute(s) |
| ml | milliliter |
| μl | microliter |
| μM | micromolar |
| μmol | micromoles |
| MC | methylcellulose |
| min | minute |
| MRM | multiple reaction monitoring |
| MS | mass spectrometry |
| MSD | MesoScale Discovery (Supplier of immunoassay kits) |
| N | Newton |
| NaCl | sodium chloride |
| NEt₃ | triethylamine |
| nM | nanomolar |
| nmol | nanomoles |
| NMR | nuclear magnetic resonance spectrometry |
| ns | not significant |
| NT | not tested |
| PAMPA | parallel artificial membrane permeability assay |
| PD | pharmacodynamic |
| Pd₂(dba)₃ | tris(dibenzylideneacetone)dipalladium(0) |
| PET | positron emission tomography |
| pg | picogram |
| PI | pharmaceutical intermediate |
| PK | pharmacokinetic |
| pmol | picomoles |
| p.o. | per os |
| q.d. or QD | quaque die |
| q.s. | quam satis |
| QC | quality control |
| Rel. | relative |
| Rf | retention factor |
| RH | relative humidity |
| rpm | revolutions per minute |
| Rt | retention time (min) |
| RT, rt | room temperature |
| SEM | standard error of the mean |
| SD | standard deviation or single dose |
| T | time |
| t_{1/2} | Half-life |
| TBME | tert-butyl-methyl-ether |
| TBS | tris-buffered saline |
| TFA | trifluoroacetic acid |
| THF | tetrahydrofuran |
| TLC | thin layer chromatography |
| Tris | tris-hydroxymethyl(aminomethane) buffer substance |
| tBu₃P | tri-tert-butyl phosphine |
| TX-100 | triton-X-100 (detergent, CAS No. 9002-93-1) |
| ULOQ | upper limit of quantification |
| UPLC | ultra performance liquid chromatography |
| vs | versus |
| WL | copper Kα radiation wavelength (λ_{Cu} = 1.5406 Å) |
| wt | weight ratio based on the quantity of starting material |
| XRPD | x-ray powder diffraction |

### Examples

The following Examples illustrate various aspects of the invention. Examples 1 and 2 show how Compound 1 may be prepared and crystallised. Examples 3, 4 and 5 describe the XRPD, DSC and stability analysis of crystalline Compound 1 (Form A). Examples 6 and 7 describe formulations comprising Compound 1 and their method of manufacture. Example 8 demonstrates the comparative stability of two formulations comprising Compound 1. Example 9 describes the dissolution profiles of formulations comprising Compound 1. Example 10 describes the dissolution profiles of Compound 1 formulations having different degrees of blend porosity. Example 11 demonstrates the relative bioavailabilities of the Experimental Formulation, Formulation A and Formulation B. Example 12 describes the lack of food effect observed in a first in human clinical study using Formulation A. Example 13 describes an in human study to assess Compound 1 PK when given administered in combination with a strong CYP3A4 inhibitor or inducer.

### Example 1: Preparation of Compound 1

The preparation of Compound 1 is described in WO 2012/095469 A1 (Example 34). Compound 1 may also be prepared as described below.

### NMR Methodology

Proton spectra are recorded on a Bruker 400 MHz ultrashield spectrometer unless otherwise noted. Chemical shifts are reported in ppm relative to methanol (b 3.31), dimethyl sulfoxide (b 2.50), or chloroform (b 7.29). A small amount of the dry sample (2-5 mg) is dissolved in an appropriate deuterated solvent (0.7 mL). The shimming is automated and the spectra obtained in accordance with procedures well known to the person of ordinary skill in the art.

### General Chromatography Information

HPLC method H1 (Rt_{H1}):
HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 30-100% B in 3.25 min, flow=0.7 ml/min

### LCMS Method H2 (Rt_{H2}):

HPLC-column dimensions: 3.0×30 mm
HPLC-column type: Zorbax SB-C18, 1.8 μm
HPLC-eluent: A) water+0.05 Vol.-% TFA, B) ACN+0.05 Vol.-% TFA
HPLC-gradient: 10-100% B in 3.25 min, flow=0.7 ml/min

### UPLCMS Method H3 (Rt_{H3}):

HPLC-column dimensions: 2.1×50 mm
HPLC-column type: Acquity UPLC HSS T3, 1.8 μm
HPLC-eluent: A) water+0.05 Vol.-% formic acid+3.75 mM ammonium acetate B) ACN+0.04 Vol.-% formic acid
HPLC-gradient: 2-98% B in 1.4 min, 98% B 0.75 min, flow=1.2 ml/min
HPLC-column temperature: 50° C.

### LCMS Method H4 (Rt_{H4}):

HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 70-100% B in 3.25 min, flow=0.7 ml/min

### LCMS Method H5 (Rt_{H5}):

HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 80-100% B in 3.25 min, flow=0.7 ml/min

### LCMS Method H6 (Rt_{H6}):

HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 40-100% B in 3.25 min, flow=0.7 ml/min

### a) 2-Bromo-5-fluoro-4-triethylsilanyl-pyridine

A solution of diisopropylamine (25.3 g, 250 mmol) in 370 ml THF was cooled with a dry-ice acetone bath at −75° C. BuLi (100 ml, 250 mmol, 2.5 M in hexanes) was added dropwise while maintaining the temperature below −50° C. After the temperature of the mixture had reached −75° C. again, a solution of 2-bromo-5-fluoropyridine (36.7 g, 208 mmol) in 45 ml THF was added dropwise. The mixture was stirred for 1 h at −75° C. Triethylchlorosilane (39.2 g, 260 mmol) was added quickly. The temperature stayed below −50° C. The cooling bath was removed and the reaction mixture was allowed to warm to −15° C., poured onto aq. NH₄Cl (10%). TBME was added and the layers were separated. The organic layer was washed with brine, dried with MgSO₄.H₂O, filtered and evaporated to give a brown liquid which was distilled at 0.5 mm Hg to yield the title compound as a slightly yellow liquid (b.p. 105-111° C.). HPLC: Rt_{H4}=2.284 min; ESIMS: 290, 292 [(M+H)⁺, 1Br]; ¹H-NMR (400 MHz, CDCl₃): 8.14 (s, 1H), 7.40 (d, 1H), 1.00-0.82 (m, 15H).

### b) 1-(6-Bromo-3-fluoro-4-triethylsilanyl-pyridin-2-yl)-ethanone

A solution of diisopropylamine (25.4 g, 250 mmol) in 500 ml THF was cooled to −75° C. BuLi (100 ml, 250 mmol, 2.5 M in hexanes) was added dropwise while maintaining the temperature below −50° C. After the reaction temperature had reached −75° C. again, a solution of 2-bromo-5-fluoro-4-triethylsilanyl-pyridine (56.04 g, 193 mmol) in 60 ml THF was added dropwise. The mixture was stirred in a dry ice bath for 70 minutes. N,N-dimethylacetamide (21.87 g, 250 mmol) was added quickly, the reaction temperature rose to −57° C. The reaction mixture was stirred in a dry ice bath for 15 min and then allowed to warm to −40° C. It was poured on a mixture of 2M aq. HCl (250 ml, 500 mmol), 250 ml water and 100 ml brine. The mixture was extracted with TBME, washed with brine, dried over MgSO₄.H₂O, filtered and evaporated to give a yellow oil which was purified on a silica gel column by eluting with hexane/0-5% TBME to yield 58.5 g of the title compound as a yellow liquid. TLC (Hex/TBME 99/1): R_{f}=0.25; HPLC: Rt_{H4}=1.921 min; ESIMS: 332, 334 [(M+H)⁺, 1Br]; ¹H-NMR (400 MHz, CDCl₃): 7.57 (d, 1H), 2.68 (s, 3H), 1.00-0.84 (m, 15H).

### c) (S)-2-(6-Bromo-3-fluoro-4-triethylsilanyl-pyridin-2-yl)-2-trimethylsilanyloxy-propionitrile

At first, the catalyst solution was prepared by dissolving water (54 mg, 3.00 mmol) in 100 ml dry DCM (≤0.001% water). This wet DCM (44 ml, 1.32 mmol water content) was added to a well stirred solution of titanium(IV) butoxide (500 mg, 1.47 mmol) in 20 ml dry DCM. The resulting clear solution was refluxed for 1 h. This solution was then cooled to rt and 2,4-di-tert-butyl-6-{[(E)-(S)-1-hydroxymethyl-2-methyl-propylimino]-methyl}-phenol [CAS 155052-31-6] (469 mg, 1.47 mmol) was added. The resulting yellow solution was stirred at rt for 1 h. This catalyst solution (0.023 M, 46.6 ml, 1.07 mmol) was added to a solution of 1-(6-bromo-3-fluoro-4-triethylsilanyl-pyridin-2-yl)-ethanone (35.53 g, 107 mmol) and trimethylsilyl cyanide (12.73 g, 128 mmol) in 223 ml dry DCM. The mixture was stirred for 2 days and evaporated to give 47 g of the crude title compound as an orange oil. HPLC: Rt_{H5}=2.773 min; ESIMS: 431, 433 [(M+H)⁺, 1Br]; ¹H-NMR (400 MHz, CDCl₃): 7.46 (d, 1H), 2.04 (s, 3H), 1.00 (t, 9H), 1.03-0.87 (m, 15H), 0.20 (s, 9H).

### d) (R)-1-Amino-2-(6-bromo-3-fluoro-4-triethylsilanyl-pyridin-2-yl)-propan-2-ol hydrochloride

Borane dimethyl sulfide complex (16.55 g, 218 mmol) was added to a solution of crude (S)-2-(6-bromo-3-fluoro-4-triethylsilanyl-pyridin-2-yl)-2-trimethylsilanyloxy-propionitrile (47 g, 109 mmol) in 470 ml THF. The mixture was refluxed for 2 h. The heating bath was removed and the reaction mixture was quenched by careful and dropwise addition of MeOH. After the evolution of gas had ceased, aq. 6M HCl (23.6 ml, 142 mmol) was added slowly. The resulting solution was evaporated and the residue was dissolved in MeOH and evaporated (twice) to yield 44.5 g of a yellow foam, pure enough for further reactions. HPLC: Rt_{H1}=2.617 min; ESIMS: 363, 365 [(M+H)⁺, 1Br]; ¹H-NMR (400 MHz, CDCl₃): 7.93 (s, br, 3H), 7.53 (d, 1H), 6.11 (s, br, 1H), 3.36-3.27 (m, 1H), 3.18-3.09 (m, 1H), 1.53 (s, 3H), 0.99-0.81 (m, 15H).

### e) (R)—N-(2-(6-bromo-3-fluoro-4-(triethylsilyl)pyridin-2-yl)-2-hydroxypropyl)-4-nitrobenzenesulfonamide

To a solution of crude (R)-1-amino-2-(6-bromo-3-fluoro-4-triethylsilanyl-pyridin-2-yl)-propan-2-01 hydrochloride (43.5 g, 109 mmol) in 335 ml THF was added a solution of NaHCO₃ (21.02 g, 250 mmol) in 500 ml water. The mixture was cooled to 0-5° C. and a solution of 4-nitrobenzenesulfonyl chloride (26.5 g, 120 mmol) in 100 ml THF was added in a dropwise. The resulting emulsion was stirred overnight while allowing the temperature to reach rt. The mixture was extracted with TBME. The organic layer was dried with MgSO₄.H₂O, filtered and evaporated to give an orange resin which was purified on a silica gel column by eluting with Hexanes/10-20% EtOAc to yield 37.56 g of the title compound as a yellow resin. TLC (Hex/EtOAc 3/1): R_{f}=0.34; HPLC: Rt_{H4}=1.678 min; ESIMS: 548, 550 [(M+H)⁺, 1Br]; ¹H-NMR (400 MHz, DMSO-d₆): 8.40 (d, 2H), 8.06 (t, 1H), 7.97 (d, 2H), 7.45 (d, 1H), 5.42 (s, 1H), 3.23 (d, 2H), 1.44 (s, 3H) 0.97-0.81 (m, 15H); Chiral HPLC (Chiralpak AD-H 1213, UV 210 nm): 90% ee.

### f) 6-Bromo-3-fluoro-2-[(S)-2-methyl-1-(4-nitro-benzenesulfonyl)-aziridin-2-yl]-4-triethylsilanyl-pyridine

A solution of triphenylphosphine (21.55 g, 82 mmol) and (R)—N-(2-(6-bromo-3-fluoro-4-(triethylsilyl)pyridin-2-yl)-2-hydroxypropyl)-4-nitrobenzenesulfonamide (37.56 g, 69 mmol) in 510 ml THF was cooled to 4° C. A solution of diethyl azodicarboxylate in toluene (40% by weight, 38.8 g, 89 mmol) was added in a dropwise while maintaining the temperature below 10° C. The cooling bath was removed and the reaction mixture was stirred at rt for 1 h. The reaction mixture was diluted with approx. 1000 ml toluene and THF was removed by evaporation at the rotavap. The resulting toluene solution of crude product was pre-purified on a silica gel column by eluting with hexanes/5-17% EtOAc. Purest fractions were combined, evaporated and crystallized from TBME/hexane to yield 29.2 g of the title compound as white crystals. HPLC: Rt_{H4}=2.546 min; ESIMS: 530, 532 [(M+H)⁺, 1Br]; ¹H-NMR (400 MHz, CDCl₃): 8.40 (d, 2H), 8.19 (d, 2H), 7.39 (d, 1H), 3.14 (s, 1H), 3.02 (s, 1H), 2.01 (s, 3H) 1.03-0.83 (m, 15H); α[D] −35.7° (c=0.97, DCM).

### g) 6-Bromo-3-fluoro-2-[(S)-2-methyl-1-(4-nitro-benzenesulfonyl)-aziridin-2-yl]-pyridine

Potassium fluoride (1.1 g, 18.85 mmol) was added to a solution of 6-bromo-3-fluoro-2-[(S)-2-methyl-1-(4-nitro-benzenesulfonyl)-aziridin-2-yl]-4-triethylsilanyl-pyridine (5 g, 9.43 mmol) and AcOH (1.13 g, 9.43 mmol) in 25 ml THF. DMF (35 ml) was added and the suspension was stirred for 1 h at rt. The reaction mixture was poured onto a mixture of sat. aq. NaHCO₃ and TBME. The layers were separated and washed with brine and TBME. The combined organic layers were dried over MgSO₄.H₂O, filtered and evaporated to give a yellow oil which was crystallized from TBME/hexane to yield 3.45 g of the title compound as white crystals. HPLC: Rt_{H6}=2.612 min; ESIMS: 416, 418 [(M+H)⁺, 1Br]; ¹H-NMR (400 MHz, CDCl₃): 8.41 (d, 2H), 8.19 (d, 2H), 7.48 (dd, 1H), 7.35 (t, 1H), 3.14 (s, 1H), 3.03 (s, 1H), 2.04 (s, 3H); α[D] −35.7° (c=0.89, DCM).

### h) (R)-2-[(R)-2-(6-Bromo-3-fluoro-pyridin-2-yl)-2-(4-nitro-benzenesulfonylamino)-propoxy]-3,3,3-trifluoro-2-methyl-propionic acid ethyl ester

A solution of (R)-3,3,3-trifluoro-2-hydroxy-2-methyl-propionic acid ethyl ester (11.93 g, 64.1 mmol) in DMF (158 ml) was evacuated/flushed with nitrogen twice. A solution of KOtBu (6.21 g, 55.5 mmol) in DMF (17 ml) was added dropwise while maintaining a reaction temperature of ca 25° C. using cooling with a water bath. After 15 min solid 6-bromo-3-fluoro-2-[(S)-2-methyl-1-(4-nitro-benzenesulfonyl)-aziridin-2-yl]-pyridine (17.78 g, 42.7 mmol) was added and stirring was continued for 3 h. The reaction mixture was poured onto a mixture of 1M HCl (56 ml), brine and TBME. The layers were separated, washed with brine and TBME. The combined organic layers were dried over MgSO₄.H₂O, filtered and evaporated. The crude reaction product was purified via chromatography on silica gel (hexanes/25-33% TBME) to yield 16.93 g of the title compound as a yellow resin that was contaminated with an isomeric side-product (ratio 70:30 by ¹H-NMR).

HPLC: Rt_{H6}=2.380 min; ESIMS: 602, 604 [(M+H)⁺, 1Br]; ¹H-NMR (400 MHz, CDCl₃): 8.32 (d, 2H), 8.07 (d, 2H), 7.46-7.41 (m, 1H), 7.30-7.23 (m, 1H), 6.92 (s, 1H), 3.39-4.30 (m, 2H), 3.95 (d, 1H), 3.84 (d, 1H), 1.68 (s, 3H), 1.56 (s, 3H), 1.40-1.34 (m, 3H)+isomeric side-product.

### i) (R)-2-[(R)-2-(6-Bromo-3-fluoro-pyridin-2-yl)-2-(4-nitro-benzenesulfonylamino)-propoxy]-3,3,3-trifluoro-2-methyl-propionamide

A solution of (R)-2-[(R)-2-(6-bromo-3-fluoro-pyridin-2-yl)-2-(4-nitro-benzenesulfonylamino)-propoxy]-3,3,3-trifluoro-2-methyl-propionic acid ethyl ester (16.93 g, 28.1 mmol) in a NH₃/MeOH (7M, 482 ml) was stirred at 50° C. in a sealed vessel for 26 h. The reaction mixture was evaporated and the residue was crystallized from DCM to yield 9.11 g of the title compound as colorless crystals.

HPLC: Rt_{H6}=2.422 min; ESIMS: 573, 575 [(M+H)⁺, 1Br]; ¹H-NMR (400 MHz, CDCl₃): 8.33 (d, 2H), 8.06 (d, 2H), 7.42 (dd, 1H), 7.30-7.26 (m, 1H), 7.17 (s, br, 1H), 6.41 (s, 1H), 5.57 (s, br, 1H), 4.15 (m, 2H), 1.68 (s, 3H), 1.65 (s, 3H).

### j) N—[(R)-1-(6-Bromo-3-fluoro-pyridin-2-yl)-2-((R)-1-cyano-2,2,2-trifluoro-1-methyl-ethoxy)-1-methyl-ethyl]-4-nitro-benzenesulfonamide

A suspension of (R)-2-[(R)-2-(6-bromo-3-fluoro-pyridin-2-yl)-2-(4-nitro-benzenesulfonylamino)-propoxy]-3,3,3-trifluoro-2-methyl-propionamide (8.43 g, 14.70 mmol) and triethylamine (5.12 ml, 36.8 mmol) in 85 ml DCM was cooled to 0-5° C. Trifluoroacetic anhydride (2.49 ml, 17.64 mmol) was added dropwise over 30 min. Additional triethylamine (1.54 ml, 11.07 mmol) and trifluoroacetic anhydride (0.75 ml, 5.29 mmol) were added to complete the reaction. The reaction mixture was quenched by addition of 14 ml aqueous ammonia (25%) and 14 ml water. The emulsion was stirred for 15 min, more water and DCM were added and the layers were separated. The organic layer was dried with MgSO₄H₂O, filtered and evaporated. Purification by column chromatography on a silica gel (hexanes/10-25% EtOAc) gave 8.09 g of the title compound as a yellow resin.

HPLC: Rt_{H6}=3.120 min; ESIMS: 555, 557 [(M+H)⁺, 1Br]; ¹H-NMR (400 MHz, CDCl₃): 8.35 (d, 2H), 8.11 (d, 2H), 7.50 (dd, 1H), 7.32 (dd, 1H), 6.78 (s, 1H), 4.39 (d 1H), 4.22 (d, 1H), 1.68 (s, 6H).

### k) (2R,5R)-5-(6-Bromo-3-fluoro-pyridin-2-yl)-2,5-dimethyl-2-trifluoromethyl-5,6-dihydro-2H-[1,4]oxazin-3-ylamine

A solution of N—[(R)-1-(6-bromo-3-fluoro-pyridin-2-yl)-2-((R)-1-cyano-2,2,2-trifluoro-1-methyl-ethoxy)-1-methyl-ethyl]-4-nitro-benzenesulfonamide (9.18 g, 16.53 mmol) and N-acetylcysteine (5.40 g, 33.10 mmol) in 92 ml ethanol was evacuated and flushed with nitrogen. K₂CO₃ (4.57 g, 33.1 mmol) was added and the mixture was stirred at 80° C. for 3 days. The reaction mixture was concentrated in vacuo to about ¼ of the original volume and partitioned between water and TBME. The organic layer was washed with 10% aq. K₂CO₃ solution, dried over Na₂SO₄, filtered and evaporated to give a yellow oil. Column chromatography on silica (hexanes/14-50% (EtOAc:MeOH 95:5)) gave 4.55 g of the title compound as an off-white solid.

HPLC: Rt_{H2}=2.741 min; ESIMS: 370, 372 [(M+H)⁺, 1Br]; ¹H-NMR (400 MHz, DMSO-d₆): 7.71-7.62 (m, 2H), 5.97 (s, br, 2H), 4.02 (d 1H), 3.70 (d, 1H), 1.51 (s, 3H), 1.47 (s, 3H).

### l) (2R, 5R)-5-(6-Amino-3-fluoro-pyridin-2-yl)-2,5-dimethyl-2-trifluoromethyl-5,6-dihydro-2H-[1,4]oxazin-3-yl amine

A glass/stainless steel autoclave was purged with nitrogen, Cu₂O (0.464 g, 3.24 mmol), ammonia (101 ml, 25%, aq., 648 mmol, 30 equivalents) and (2R,5R)-5-(6-Bromo-3-fluoro-pyridin-2-yl)-2,5-dimethyl-2-trifluoromethyl-5,6-dihydro-2H-[1,4]oxazin-3-ylamine (8 g, 21.6 mmol) in ethylene glycol (130 ml) was added. The autoclave was closed and the suspension heated up to 60° C. and the solution was stirred for about 48 hours (max. pressure 0.7 bar, inside temperature 59-60° C.). The reaction mixture was diluted with ethyl acetate and water. The organic phase was washed with water and 4 times with 12% aq. ammonia and finally with brine, dried over sodium sulfate, filtered and evaporated. The crude product (7 g, containing some ethylen glycol, quantitative yield) was used in the next step without further purification.

HPLC: Rt_{H3}=0.60 min; ESIMS: 307 [(M+H)⁺].

### m) [(2R, 5R)-5-(6-Amino-3-fluoro-pyridin-2-yl)-2,5-dimethyl-2-trifluoromethyl-5,6-dihydro-2H-[1,4]oxazin-3-yl]-carbamic acid tert-butyl ester

A solution of (2R, 5R)-5-(6-amino-3-fluoro-pyridin-2-yl)-2,5-dimethyl-2-trifluoromethyl-5,6-dihydro-2H-[1,4]oxazin-3-yl amine (6.62 g, 21.6 mmol), Boc₂O (4.72 g, 21.6 mmol) and Hunig's base (5.66 ml, 32.4 mmol) in dichloromethane (185 ml) was stirred at rt for 18 hours. The reaction mixture was washed with sat. aq. NaHCO₃ and brine. The aqueous layers were back extracted with dichloromethane and the combined organic layers were dried over sodium sulfate, filtered and evaporated to give a light green solid (14 g). The crude product was chromatographed over silica gel (cyclohexane:ethyl acetate 95:5 to 60:40) to afford 7.68 g of the title compound.

TLC (cyclohexane:ethyl acetate 3:1): R_{f}=0.21; HPLC: Rt_{H3}=1.14 min; ESIMS: 408 [(M+H)⁺]; ¹H-NMR (400 MHz, CDCl3): 11.47 (br. s, 1H), 7.23 (dd, J=10.42, 8.78 Hz, 1H), 6.45 (dd, J=8.78, 2.64 Hz, 1H), 4.50 (br. s, 2H), 4.32 (d, J=2.38 Hz, 1H), 4.10 (d, J=11.80 Hz, 1H), 1.69 (s, 3H, CH3), 1.65 (s, 3H, CH3), 1.55 (s, 9H).

### n) ((2R, 5R)-5-{6-[(3-Chloro-5-trifluoromethyl-pyridine-2-carbonyl)-amino]-3-fluoro-pyridin-2-yl}-2,5-dimethyl-2-trifluoromethyl-5,6-dihydro-2H-[1,4]oxazin-3-yl)-carbamic acid tert-butyl ester

A mixture of [(2R, 5R)-5-(6-amino-3-fluoro-pyridin-2-yl)-2,5-dimethyl-2-trifluoromethyl-5,6-dihydro-2H-[1,4]oxazin-3-yl]-carbamic acid tert-butyl ester (3.3 g, 8.12 mmol), 3-chloro-5-trifluoromethylpicolinic acid (2.2 g, 9.74 mmol), HOAt (1.99 g, 14.62 mmol) and EDC hydrochloride (2.33 g, 12.18 mmol) was stirred in DMF (81 ml) at rt for 48 hours. The reaction mixture was diluted with ethyl acetate and washed with water and brine, dried over sodium sulfate, filtered and evaporated. The crude product (12 g) was chromatographed over silica gel (cyclohexane to cyclohexane:ethyl acetate 1:1) to yield 5.2 g of the title compound.

TLC (silica, cyclohexane:ethyl acetate 3:1): R_{f}=0.47; HPLC: Rt_{H3}=1.40 min; ESIMS: 615, 616 [(M+H)⁺, 1Cl]; ¹H-NMR (400 MHz, CDCl₃): 11.68 (s, 1H), 10.41 (s, 1H), 8.81 (dd, J=1.82, 0.69 Hz, 1H), 8.45 (dd, J=8.91, 3.14 Hz, 1H), 8.19 (dd, J=1.88, 0.63 Hz, 1H), 7.59 (dd, J=9.79, 9.16 Hz, 1H), 4.38 (d, J=2.13 Hz, 1H), 4.18 (d, J=11.80 Hz, 1H), 1.75 (s, 3H), 1.62 (s, 3H), 1.60 (s, 9H).

### o) 3-Chloro-5-trifluoromethyl-pyridine-2-carboxylic acid [6-((3R,6R)-5-amino-3,6-dimethyl-6-trifluoromethyl-3,6-dihydro-2H-[1,4]oxazin-3-yl)-5-fluoro-pyridin-2-yl]-amide

A mixture of ((2R, 5R)-5-{6-[3-chloro-5-trifluoromethyl-pyridine-2-carbonyl)-amino]-3-fluoro-pyridin-2-yl}-2,5-dimethyl-2-trifluoromethyl-5,6-dihydro-2H-[1,4]oxazin-3-yl)-carbamic acid tert-butyl ester (4.99 g, 8.13 mmol) and TFA (6.26 ml, 81 mmol) in dichloromethane (81 ml) was stirred at rt for 18 hours. The solvent was evaporated and the residue diluted with a suitable organic solvent, such as ethyl acetate and aq. ammonia. Ice was added and the organic phase was washed with water and brine, dried over sodium sulfate, filtered and evaporated to yield 3.78 g of the title compound.

HPLC: Rt_{H3}=0.87 min; ESIMS: 514, 516 [(M+H)⁺, 1Cl]; ¹H-NMR (400 MHz, DMSO-d₆): δ 11.11 (s, 1H), 9.06 (s, 1H), 8.69 (s, 1H), 8.13 (dd, J=8.8, 2.6 Hz, 1H), 7.80-7.68 (m, 1H), 5.88 (br. s, 2H), 4.12 (d, J=11.5 Hz, 1H), 3.72 (d, J=11.4 Hz, 1H), 1.51 (s, 3H), 1.49 (s, 3H).

### Example 2: Crystallisation Procedure for Compound 1

1 wt of Compound 1 was dissolved in 5.11 wt of IPAc at 70-80° C. The solution was filtered (filter <2 μm) and then 1.52 wt of n-heptane added. The solution was cooled to 55° C., and seeded with 0.5% w/w of Compound 1. The suspension was held at 55° C. for 30-60 mins and then cooled to 35° C. over 2 hours. The suspension was aged for 1 hour and then 8.2 wt of n-heptane were added over 3 hours. The suspension was aged for 1 hour and then cooled to 0-5° C. over 2 hours and aged for at least 2 hours. The suspension was filtered under vacuum, and the cake washed with 10/90 w/w isopropyl acetate/n-heptane. The cake was dried under vacuum at 40-45° C. until dry.

### Example 3: XRPD Analysis of Crystalline Compound 1

Crystalline Compound 1 was analysed by XRPD and the ten most characteristic peaks are shown in Table 1 (see also FIG. 1).

TABLE 1

| 2-theta in degrees | d-value in Å | relative intensity in % |
|---|---|---|
| 10.68 | 8.28 | 67.4 |
| 14.84 | 5.96 | 100.0 |
| 18.66 | 4.75 | 23.5 |
| 19.52 | 4.54 | 46.6 |
| 21.38 | 4.15 | 71.4 |
| 21.68 | 4.10 | 19.9 |
| 25.52 | 3.49 | 5.4 |
| 29.86 | 2.99 | 6.8 |
| 35.04 | 2.56 | 6.0 |
| 37.83 | 2.38 | 4.5 |

X-ray powder diffraction (XRPD) analysis was performed using a Bruker D8 Advance x-ray diffractometer in reflection geometry. Measurements were taken at about 30 kV and 40 mA under the following conditions:

TABLE 2

| | |
|---|---|
| Scan rate (continuous scan): | 3 s/step |
| Step size: | 0.017° (2-theta) |
| Soller slit: | 2.5° |
| Slits (from left to right): | V12 (variable) |

The X-ray diffraction pattern was recorded between 2° and 40° (2-theta) with CuK_{α} radiation for identification of the whole pattern.

### Example 4: DSC Analysis of Crystalline Compound 1

Crystalline Compound 1 was analysed by differential scanning calorimetry (DSC) using a Q1000 Diffraction Scanning Calorimeter from TA instruments and found to have an onset of melting at about 171° C., see FIG. 2.

### Example 5: Chemical Stability of Crystalline Compound 1 when Exposed to High Temperature/Humidity for One Week

The stability of crystalline Compound 1 was tested by exposing the crystalline material to high temperature and/or humidity for at least three weeks. After storage at high temperature and/or humidity, bulk crystalline material was sampled and dissolved in acetonitrile:water (80:20) and the purity analysed in a Waters Aquity UPLC using the following conditions:

TABLE 3

| Separation column | Waters Acquity UPLC BEH Phenyl | | |
|---|---|---|---|
| Mobile phase | A: 0.05% TFA in 95% water/5% acetonitrile; B: 0.05% TFA in | | |
| | 95% acetonitrile/5% water | | |
| Flow rate | 0.6 mL/min | | |
| Column Temperature ° | 35° C. | | |
| Detection | 286 nm | | |
| Gradient | Time (min) | % A | % B |
| | 0.0 | 95 | 5 |
| | 2.5 | 60 | 40 |
| | 3.5 | 54 | 46 |
| | 5.0 | 5 | 95 |
| | 5.01 | 95 | 5 |
| | 6.0 | 95 | 5 |

The results of this test are shown in Table 4 below.

TABLE 4

| Test Conditions | | |
|---|---|---|
| Temp/RH; Exposure Time | Purity/% | Solid State Form |
| RT; 0 | 97.3 | Crystalline |
| 80° C.; 3 weeks | 97.3 | Crystalline |
| 50° C.; 4 weeks | 97.3 | Crystalline |
| 50° C./75% RH; 3 weeks | 96.8 | Crystalline |

This crystalline form “Form A” is the most stable of the free base forms of Compound 1 discovered.

### Example 6: Pharmaceutical Composition Comprising Compound 1—Formulation ‘A’

Compound 1 was formulated as 1, 10, 25, and 75 mg dose strength hard gelatin capsules (e.g. Capsugel, size 3) comprising the ingredients shown in Table 5 (Formulation A). Batch manufacturing was carried out as described below and in Table 6.

TABLE 5
Composition of 1 mg, 10 mg, 25 mg and 75 mg Compound
1 hard gelatin capsule (Formulation A)

| | Formulation A amount per capsule (% w/w) | | | |
|---|---|---|---|---|
| | 1 mg | 10 mg | 25 mg | 75 mg |
| Drug load | 0.6% | 5.9% | 14.7% | 44.1% |
| Capsule fill ingredient | | | | |
| Compound 1 | 0.6 | 5.9 | 14.7 | 44.1 |
| Mannitol | 67.2 | 63.37 | 56.91 | 35.14 |
| Pregelatinized starch | 21.77 | 20.29 | 17.94 | 10.29 |
| Low substituted hydroxypropyl cellulose | 5.17 | 5.17 | 5.18 | 5.17 |
| hydroxypropyl cellulose | 3.39 | 3.39 | 3.39 | 3.39 |
| Talc | 0.47 | 0.47 | 0.47 | 0.47 |
| Sodium stearyl fumarate | 1.41 | 1.41 | 1.41 | 1.41 |
| Weight capsule fill mix (mg) | 170.0 | 170.0 | 170.0 | 170.0 |

TABLE 6
Manufacturing of 1 mg, 10 mg, 25 mg and 75 mg hard
gelatin capsules of Compound 1 (Formulation A)

| | Amount per batch (kg) | | | |
|---|---|---|---|---|
| | 1 mg | 10 mg | 25 mg | 75 mg³ |
| Batch size | 7500 | 16,000 | 35,000 | 7,100 |
| | units | units | units | units |
| Drug load | 0.6% | 5.9% | 14.7% | 44.1% |
| Capsule fill ingredient | | | | |
| Compound 1¹ | 0.0075 | 0.1600 | 0.875 | 0.5325 |
| Mannitol | 0.8568 | 1.7238 | 3.386 | 0.4242 |
| Pregelatinised starch | 0.2775 | 0.5520 | 1.068 | 0.1243 |
| Low-substituted Hydroxypropyl | 0.0660 | 0.1408 | 0.308 | 0.0625 |
| cellulose | | | | |
| Hydroxypropylcellulose | 0.0432 | 0.0922 | 0.202 | 0.0409 |
| Sodium stearyl fumarate | 0.0180 | 0.0384 | 0.084 | 0.0170 |
| Talc | 0.0060 | 0.0128 | 0.028 | 0.0057 |
| Purified water² | q.s. | q.s. | q.s. | q.s. |
| Weight capsule fill mix | 1.2750 | 2.7200 | 5.950 | 1.2071 |
| Empty capsule shell | | | | |
| Capsule shell, size 3 (theoretical | 0.3600 | 0.7680 | 1.680 | 0.3408 |
| weight) | | | | |
| Total batch weight | 1.6350 | 3.4880 | 7.630 | 1.5479 |

¹Corresponding to a corrected drug substance content (=cc) of 100%. A compensation of drug substance is performed if the corrected drug substance content is ≤99.5%. The difference in weight is adjusted with Mannitol.
²Removed during processing
³During granulation of the 75 mg strength formulation, it was observed that the granulation process was inadequate. This is likely attributed to the high drug load of 44% w/w in this composition. Therefore, for reliable granulation process, an upper limit to the drug load of, for example, 35% should be maintained.

Other batch sizes may be prepared depending on supply requirements and/or available equipment chain. The weight of individual components for other batch sizes corresponds proportionally to the stated composition.

### Description of Manufacturing Process of Compound 1 Formulation A: 1 mg and 10 mg Hard Gelatin Capsules

1. Blend drug substance Compound 1 and portion of mannitol.
2. Sieve the mixture of step 1.
3. Blend the mixture of step 2.
4. Sieve portion of mannitol and add to the mixture of step 3.
5. Blend the mixture of step 4.
6. Sieve remaining portion of mannitol, pre-gelatinised starch, low-substituted hydroxypropyl cellulose and hydroxypropyl cellulose. Add the sieved ingredients to the mixture of step 5.
7. Blend the mixture of step 6.
8. Sieve the blend of step 7.
9. Blend the mixture of step 8.
10. Dissolve hydroxypropyl cellulose in purified water under stirring to form binder solution. Add binder solution to the blend of step 9 and granulate the mass using a high shear granulator (for example Collette).
11. Perform wet screening of mass from step 10 if necessary.
12. Dry the wet granules of step 11 in a fluid bed drier (for example Aeromatic).
13. Screen the dried granules of step 12.
14. Sieve mannitol, low-substituted hydroxypropyl cellulose and talc and add to the sieved granules of step 13.
15. Blend the mixture of step 14.
16. Sieve sodium stearyl fumarate and add to mixture of step 15.
17. Blend the mixture of step 16 to get final blend.
18. Encapsulate the final blend from step 17 using capsule filling machine (for example H&K).

### Description of Manufacturing Process of Compound 1 Formulation A: 25 mg and 75 mg Hard Gelatin Capsules

1. Sieve drug substance Compound 1, mannitol, pre-gelatinised starch, low substituted hydroxypropyl cellulose, hydroxypropyl cellulose.
2. Blend the sieved materials of step 1.
3. Sieve the mixture of step 2.
4. Blend the mixture of step 3.
5. Dissolve hydroxypropyl cellulose in purified water under stirring to form binder solution. Add binder solution to the blend of step 4 and granulate the mass using a high shear granulator (for example Collette).
6. Perform wet screening of mass from step 6 if necessary
7. Dry the wet granules of step 6 in a fluid bed drier (for example Aeromatic).
8. Screen the dried granules of step 7.
9. Sieve mannitol, low-substituted hydroxypropyl cellulose and talc and add to sieved granules of step 8.
10. Blend the mixture of step 9.
11. Sieve sodium stearyl fumarate and add to step 10.
12. Blend the mixture of step 11 to get final blend.
13. Encapsulate the final blend of step 12.

The processes described above may be reasonably adjusted depending on the available equipment chain and batch scale. Different batch sizes can be prepared by adaptation of equipment size. The weight of individual components for other batch sizes corresponds proportionally to the stated composition within the usual adaptation that may be needed to enable process scale up and transfer as depicted for example in FDA guidance on scale-up and post approval changes.

### Example 7: Further Pharmaceutical Composition Comprising Compound 1—Formulation ‘B’

Compound 1 was additionally formulated as a hard gelatin capsule (e.g. Capsugel, size 2 or 3) comprising the ingredients shown in Table 7 (Formulation B). Formulation B manufacture was carried out as described below and in Table 8.

TABLE 7
Unit composition of 10 mg, 15 mg, 25 mg and 50 mg dose strength
formulations of Compound 1 hard gelatin capsules (Formulation B)

| | Formulation B Amount per capsule | | | |
|---|---|---|---|---|
| | (% w/w) | | | |
| | 10 mg | 15 mg | 25 mg | 50 mg |
| Drug load | 8.3% | 8.3% | 20.8% | 20.8% |
| Capsule fill ingredient | | | | |
| Compound 1 | 8.33¹ | 8.33¹ | 20.83¹ | 20.83¹ |
| Mannitol² | 42.97³ | 42.97⁴ | 39.30⁵ | 39.30⁶ |
| Microcrystalline cellulose | 38.83 | 38.83 | 30.00 | 30.00 |
| Low substituted hydroxypropyl | 5.00 | 5.00 | 5.00 | 5.00 |
| cellulose | | | | |
| Hypromellose | 2.87 | 2.87 | 2.87 | 2.87 |
| Sodium stearyl fumarate | 1.50 | 1.50 | 1.50 | 1.50 |
| Talc | 0.50 | 0.50 | 0.50 | 0.50 |
| Purified water⁷ | — | — | — | — |
| Capsule fill weight (mg) | 120.00 | 180.00 | 120.00 | 240.00 |
| Empty capsule shell (theoretical | 48.00⁸ | 61.00⁹ | 48.00⁸ | 61.00⁹ |
| weight in mg) | | | | |
| Total capsule weight (mg) | 168.00 | 241.00 | 168.00 | 301.00 |

¹Formulation B uses a co-milled blend of 50% w/w drug substance and 50% w/w mannitol
²Total mannitol amount in the formulation including mannitol from co-milled blend (pharmaceutical intermediate—PI) and mannitol added in blend for granulation.
³Includes 10.000 mg (8.33% w/w) from co-milled blend and 41.560 mg (34.63% w/w) taken in blend for granulation
⁴Includes 15.000 mg (8.33% w/w) from co-milled blend and 62.340 mg (34.63% w/w) taken in blend for granulation
⁵Includes 25.000 mg (20.83% w/w) from co-milled blend and 22.160 mg (18.47% w/w) taken in blend for granulation
⁶Includes 50.000 mg (20.83% w/w) from co-milled blend and 44.320 mg (18.47% w/w) taken in blend for granulation
⁷Removed during procesing
⁸Formulation B 10 mg (8.33% w/w) and 25 mg (20.83% w/w) dosage strengths are filled in size 3 hard gelatin capsules
⁹Formulation B 15 (8.33% w/w) and 50 mg (20.83% w/w) dosage strength is filled in size 2 hard gelatin capsules

In Formulation B, the drug substance Compound 1 and mannitol are co-milled in order to improve robustness of the milling process. Milling of neat drug substance was found to be challenging due to poor flow and sticking tendency of the material. Examples of suitable mills for the co-milling process include, but are not limited to, Hosokawa Alpine mills, for example: AS, AFG and JS system models; or Fluid Energy Processing & Equipment Company mills, for example: Roto-Jet system models. The co-milled blend is considered as a pharmaceutical intermediate (PI) that is further processed to manufacture the drug product. The co-milled blend utilized in Formulation B contains 50% w/w drug substance Compound 1 and 50% w/w mannitol. Lab scale development trials and small scale pilot manufacturing of co-milled blend containing drug substance Compound 1 up to 70% w/w and mannitol up to 30% w/w (i.e. 70:30—drug substance Compound 1: mannitol) led to a cumbersome process due to poor material properties of the blend and adherence to the milling chamber. Co-milling of drug substance Compound 1 with 15% w/w mannitol failed. The 50:50% w/w (or 1:1) ratio of drug substance Compound 1 to mannitol was subsequently used based on the positive readout of a manufacturing trial at this ratio.

Formulations A and B are produced by wet granulation technology. Wet granulation was chosen to overcome challenging drug substance physical properties, namely low bulk density, poor flow and wettability. Pregelatinized starch and hydroxypropyl cellulose used as filler and binder respectively in Formulation A were replaced by microcrystalline cellulose and hypromellose. Experiments showed that use of microcrystalline cellulose as filler, rather than pregelatinized starch, led to a faster dissolution profile and improved granule properties. Further experiments showed that use of hypromellose as binder, rather than hydroxypropyl cellulose, provided improved granule properties and granulation process.

TABLE 8
Manufacturing formula for Compound 1 Formulation B:
10 mg, 15 mg, 25 mg and 50 mg hard gelatin capsules

| | Amount per batch (kg) | | | |
|---|---|---|---|---|
| | Formulation B dose strength and batch size | | | |
| | 10 mg, | 15 mg, | 25 mg, | 50 mg, |
| | 40,000 | 255,650 | 40,000 | 219,000 |
| Ingredient | capsules | capsules | capsules | capsules |
| Capsule fill | | | | |
| Compound 1 PI¹ | 0.800 | 7.670 | 2.000 | 21.900 |
| Microcrystalline | 1.864 | 17.870 | 1.440 | 15.768 |
| cellulose | | | | |
| Mannitol | 1.662 | 15.937 | 0.886 | 9.706 |
| Low substituted | 0.240 | 2.301 | 0.240 | 2.628 |
| hydroxypropyl cellulose | | | | |
| Hypromellose | 0.138 | 1.319 | 0.138 | 1.507 |
| Sodium stearyl fumarate | 0.072 | 0.690 | 0.072 | 0.788 |
| Talc | 0.024 | 0.230 | 0.024 | 0.263 |
| Purified water² | q.s | q.s | q.s | q.s |
| Weight capsule fill mix | 4.800 | 46.017 | 4.800 | 52.560 |
| Empty capsule shell | 1.920 | 15.595 | 1.920 | 13.359 |
| Capsule shell³ | | | | |
| (theoretical weight) | | | | |
| Total batch weight | 6.720 | 61.612 | 6.720 | 65.919 |

¹If PI drug content is ≤99.5% or ≥100.5%, the weight will be adjusted and compensated with mannitol
²Removed during processing
³10 and 25 mg dose strength blends were filled into Size 3 hard gelatin capsules whereas 15 and 50 mg does strength blends were filled into Size 2 hard gelatin capsules
q.s = quantum satis (to be added as needed)

Table 8 provides the ingredients for particular batch sizes. Other batch sizes may be utilised depending on clinical requirements and/or available equipment and/or available starting materials. The weight of individual components for other batch sizes corresponds proportionally to the stated composition.

### Description of Manufacturing Process

The process described below may be reasonably adjusted, while maintaining the same basic production steps, to compensate for different batch sizes and/or equipment characteristics, and/or on the basis of experience of the previous production batch.

### PI Manufacture

1. Blend drug substance Compound 1 and mannitol.
2. Sieve the blend of step 1.
3. Co-mill the sieved material of step 2.
4. Blend the co-milled material of step 3 to obtain Compound 1 PI

### Compound 1 Formulation B: 15 mg and 50 mg Hard Gelatin Capsules

1. Sieve Compound 1 PI, mannitol, microcrystalline cellulose, and low substituted hydroxypropyl cellulose.
2. Blend the sieved materials of step 1.
3. Sieve the mixture of step 2.
4. Blend the mixture of step 3.
5. Dissolve hypromellose in purified water under stirring to form binder solution. Add binder solution to the blend of step 4 and granulate the mass using a high shear granulator (for example Collette Model GRAL). Add additional purified water if necessary. Target amount of total water: approximately 25%.
6. Perform wet screening based on visual observation/assessment of wet granules of step 5 (optional).
7. Dry the wet granules of step 6 in a fluid bed dryer (for example Aeromatic).
8. Screen the dried granules of step 7.
9. Sieve low-substituted hydroxypropyl cellulose and talc and add to sieved granules of step 8.
10. Blend the mixture of step 9.
11. Sieve sodium stearyl fumarate and add to step 10.
12. Blend the mixture of step 11 to get final blend.
13. Encapsulate the final blend of step 12 into hard gelatin capsules.

### Example 8: Comparative Stability of Compound 1 in Formulation A and B Hard Gelatin Capsules

A first batch set of Compound 1 Formulation A: 1 mg, 10 mg and 75 mg hard gelatin capsules, stored in HDPE bottle, was found to be stable at 40° C./75% RH for 1 month for the 1 mg dosage strength and up to 6 months for the 10 and 75 mg dosage strengths. These stability results support a shelf-life of 24 months at long term storage “Store at 2-8° C.” in HDPE bottle.

The 3 months compliant stability results of Compound 1 Formulation B: 15 mg and 50 mg hard gelatin capsules at 25° C./60% RH in open bottle and under accelerated conditions (40° C./75% RH) support a shelf-life of 12 months at “do not store above 25° C.” long term storage in HDPE bottles, i.e. no refrigeration required.

The results of the comparative stability study of Compound 1 in Formulations A and B stored in high density polyethylene bottles (175 ml), in terms of percentage total degradation products, are summarised in Table 9 below. Total degradation products were measured by HPLC.

TABLE 9
Comparative stability of Compound 1 in Formulations A and B

| | | | Formulation Type | | | | | | | |
|---|---|---|---|---|---|---|---|---|---|---|
| | | | A | | | | B | | | |
| | | | Capsule Strength | | | | | | | |
| | | | 1 mg | 10 mg | 25 mg | 75 mg | 10 mg | 15 mg | 25 mg | 50 mg |
| Drug Load % w/w¹ | | | 0.6 | 5.9 | 14.7 | 44.1 | 8.3 | 8.3 | 20.8 | 20.8 |
| | | | Total Degradation Products [%] | | | | | | | |
| Storage | | | Initial | | | | | | | |
| Conditions | Time Point | | 0.3 | 0.3 | <0.1 | 0.3 | <0.1 | <0.1 | <0.1 | <0.1 |
| 25° C./60% | 1 | month | 0.3 | 0.2 | <0.1 | 0.2 | <0.1 | NT | <0.1 | NT |
| RH | 6 | weeks | 0.6 | 0.4 | NT | 0.3 | NT | <0.1 | NT | <0.1 |
| | 3 | months | 0.4 | 0.3 | <0.1 | 0.2 | <0.1 | <0.1 | <0.1 | <0.1 |
| | 6 | months | 0.9 | 0.4 | <0.1 | 0.3 | <0.1 | <0.1 | <0.1 | <0.1 |
| | 12 | months | 1.2 | 0.4 | <0.1 | 0.3 | NT | NT | NT | NT |
| 40° C./75% | 1 | month | 1.4 | 0.4 | NT | 0.3 | NT | NT | NT | NT |
| RH | 6 | weeks | NT | NT | NT | NT | NT | <0.1 | NT | <0.1 |
| | 3 | months | 3.9 | 0.8 | 0.2 | 0.3 | 0.4 | 0.1 | 0.1 | <0.1 |
| | 6 | months | 10.6 | 1.6 | 0.5 | 0.4 | 0.8 | 0.6 | 0.4 | 0.2 |

NT = Not Tested
¹Percentage mass of the drug substance/capsule fill weight in the absence of the empty capsule shell weight

The data in Table 10 demonstrate that Formulation B (10-50 mg dosage strength) is more stable than Formulation A (1-75 mg dosage strength) and that drug product stability is improved with increasing drug load.

### Example 9: Dissolution Comparisons of Experimental Formulation and Formulations A and B

An experimental formulation (EF) based on drug in capsule approach was developed to support in-vitro in-vivo correlation (IVIVC) modelling. In the preparation of the EF, Compound 1 was co-milled with mannitol such that 1 g PI contained 700 mg of Compound 1, i.e. a co-milled blend of 70% w/w drug substance and 30% w/w mannitol. Co-milled drug substance Compound 1 was filled into HGCs to provide a 25 mg dosage strength EF (35.73 mg/unit composition).

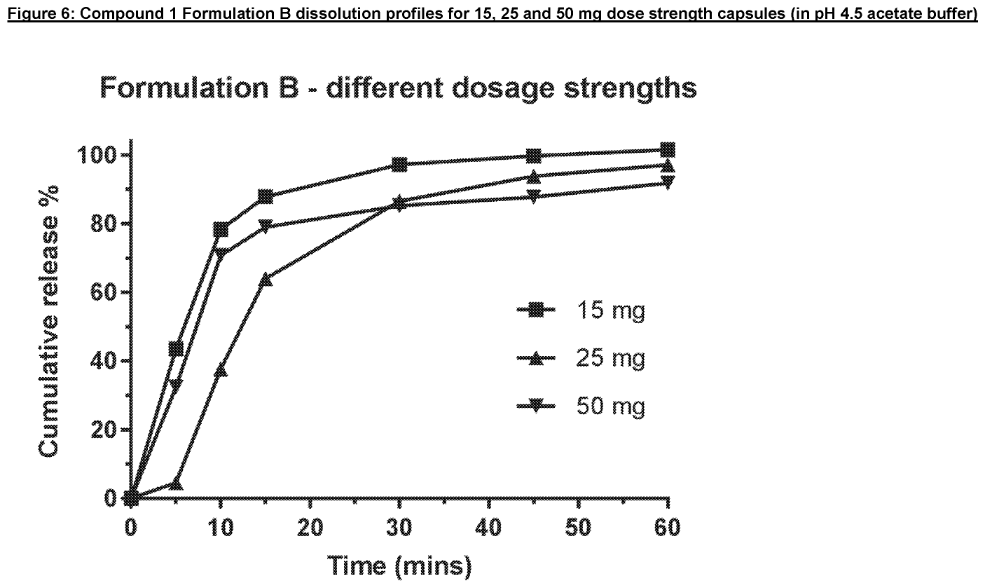

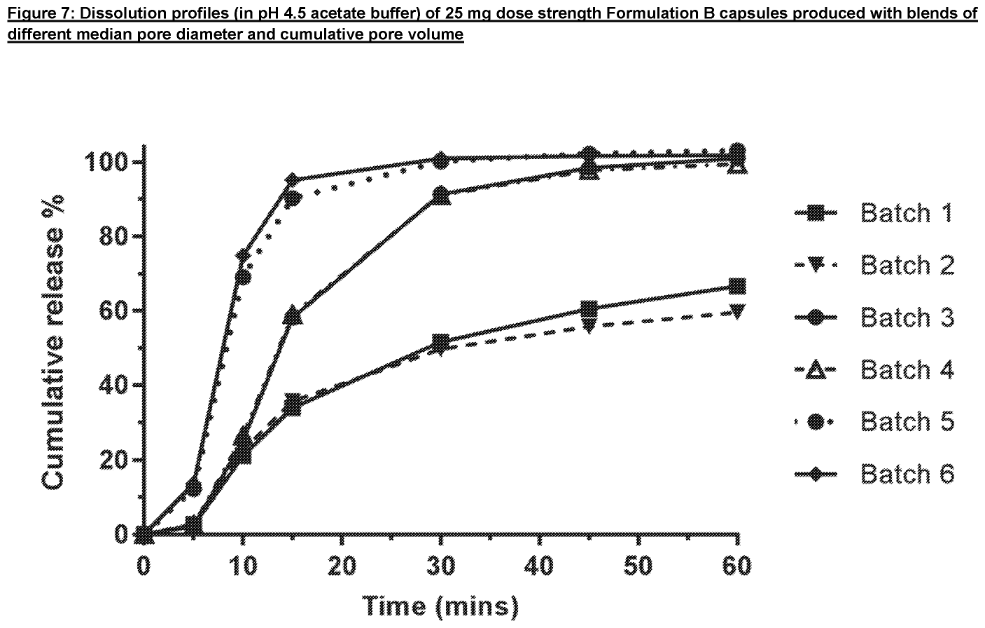

The amount of drug substance dissolved in a dissolution apparatus (basket method described in US Pharmacopeia Chapter <711> “Dissolution”), edition 39-NF 34, was determined by UV detection and dissolution profiles created for the Experimental Formulation (EF) and Formulations 1 (FA) and 2 (FB) in the following test media: 0.01N HCl; 0.1N HCl; acetate buffer pH 4.5; fasted state simulated intestinal fluid (FaSSIF; Klein S, 2010); and fed state simulated intestinal fluid (FeSSIF; Klein S, 2010). A summary of the method is provided in Table 10 below and the results shown in FIGS. 3, 4 and 5, for the EF, FA and FB respectively. The dissolution profiles of the 15, 25 and 50 mg dose strength Formulation B capsules in acetate buffer pH 4.5 are shown in FIG. 6. These results demonstrate the improved dissolution profile, in terms of rate and extent of dissolution, of FA and FB in comparison to EF, particularly at the biologically relevant pH 4.5 (see Example 11). Slightly slower dissolution profile of 25 mg in FIG. 6 compared to 15 mg and 50 mg at initial time points is understood to be stemming from the delay in the gelatin dissolution and capsule opening.

TABLE 10
Dissolution determination by UV

| Principle | Measurement of the amount of drug |
|---|---|
| | substance dissolved in a dissolution apparatus |
| | 1 (basket) according to USP <711> |
| | “Dissolution”. Determination by |
| | UV detection. |
| Reagents | |
| Methanol | Gradient grade, e.g. Merck No. 1.06007 |
| Water | Demineralized (purified), |
| | e.g from Millipore Q |
| Sodium acetate | ACS grade, e.g. Merck 1.06235 |
| trihydrate | |
| Acetic acid 100% | ACS grade, e.g. Merck 1.00063 |
| Dissolution | Basket method according to |
| conditions | USP <711>, “Dissolution” |
| Speed of rotation | 100 ± 2 rpm |
| 2N Acetic | Example of preparation: |
| acid solution | Dilute 58 mL of acetic acid (100%) to 500 mL |
| | with deionized water. Mix well. |
| Test medium^{1,2} | Acetate buffer pH 4.5. Example of |
| | preparation: weigh accurately 30.0 g of sodium |
| | acetate trihydrate, add 140 mL of 2N acetic acid |
| | solution and complete to 10 L with deionized |
| | water. Stir until dissolved, measure the pH, |
| | adjust to pH 4.5 if needed with 2N |
| | acetic acid solution. |
| Volume of | 500 mL up to 15 mg Compound |
| test medium | 1 dosage strength |
| | 900 mL over 15 mg Compound |
| | 1 dosage strength |
| Temperature | 37 ± 0.5° C. |
| Evaluation | Determine the absorbance of the test |
| | solutions using a suitable spectrophotometer, |
| | for example Evolution 201 or 220 |
| | UV-Visible Spectrophotometer |
| | (ThermoFisher Scientific) |
| UV parameter | |
| conditions | |
| Cell (Quartz) | 1.0 cm |
| Blank/Reference | Test medium |
| Wavelength(s) | UV 283 nm |
| Calculation, using the A1% | ${D}_{{un}_{i}} = \frac{{AT}_{i} \times {V}_{T} \times 10 \times 100}{A 1 \% \times {m}_{D} \times d \times SF}$ |
| | $A 1 \% = \frac{AR \times {V}_{R} \times 10 \times 100}{{m}_{R} \times {C}_{R} \times d}$ |
| | Standard value for Compound |
| | 1 = 248.9 (245.2 − 252.6) |
| Where | |
| n | Number of sampling points |
| Dᵤₙ | Dissolution of Compound 1, in percentage |
| | of the declared content uncorrected |
| | regarding the volume withdrawn. |
| Dᵤₙ_{ᵢ} | Each of the individual Dᵤₙ at the |
| | respective sampling time points, |
| | indexed by i |
| i | Running factor for indexing the sampling |
| | time points. It starts with 1 for the first |
| | sampling time point and ends with n for the |
| | last considered sampling point. |
| A1% | Specific absorbance of a 1% (m/v) solution at |
| | 283 nm normalized to a cell path of 1.0 cm |
| ATᵢ | Absorbance of Compound 1 at the absorbance |
| | maxima at about 283 nm in the test solution |
| | at sampling time point i. |
| m_{R} | Mass of reference substance in mg |
| V_{T} | Volume of the test solution in ml |
| C_{R} | Declared content of the reference substance |
| | in percent |
| AR | Absorbance of Compound 1 at the absorbance |
| | maxima at about 283 nm in the |
| | reference solution. |
| V_{R} | Volume of the reference solution in ml |
| m_{D} | Declared drug substance content in mg |
| | per dosage form |
| SF | Salt/base factor (1.000) |
| d | Cell thickness in cm |
| 10 | Conversion factor mg/ml to percent |
| 100 | Conversion factor to percent |

¹1 litre of FaSSIF medium is prepared by (Step 1, preparation of maleate buffer) dissolving: 1.39 g NaOH (pellets); 2.23 g of maleic acid; 4.01 g of NaCl; in 0.9 L of purified water and adjusting the pH to 6.5 with either 1N NaOH or 1N HCl and making up to volume (1 L) with purified water. (Step 2) adding 1.79 g of FaSSIF-V2 powder (biorelevant.com, London, United Kingdom) to about 500 ml of maleate buffer at room temperature, stirring until powder has dissolved, making up to volume of (1 L) with the buffer and letting the medium stand for 1 hour.
²1 litre of FeSSIF medium is prepared by (Step 1, preparation of maleate buffer) dissolving 3.27 g NaOH (pellets); 6.39 g of maleic acid; and 7.33 g of NaCl in 0.9 L of purified water and adjusting the pH to 5.8 with either 1N NaOH or 1N HCl and making up to volume (1 L) with purified water. (Step 2) adding 9.76 g of FeSSIF-V2 (biorelevant.com, London, United Kingdom) powder to about 500 ml of buffer at room temperature, stirring until powder has dissolved, making up to volume (1 L) with the buffer and letting the medium stand for 1 hour.

### Example 10: Dissolution Profiles of Formulations Produced with Blends of Different Median Pore Diameter and Cumulative Pore Volume

Six separate batches of Formulation B, 25 mg Compound 1 dose strength (batches 1 to 6 in Table 11 below) were prepared as described previously in Example 7 using a lab scale granulator (for example Collette Gral 10L). The percentage of water used during wet granulation, the impeller speed, and duration of wet granulation, were varied between the batches as set out below in Table 11. Additionally, one batch each of 15 and 50 mg, batches 7 and 8 respectively, were produced using a pilot scale granulator (for example Collette Gral 75L). The corresponding parameters are also listed in Table 11.

TABLE 11
Formulation B batch wet granulation parameters

| | | Wet granulation parameters | | | |
|---|---|---|---|---|---|
| | | Total amount | Granulation | | |
| | | of water used | duration − | | |
| | | (% w/w of | binder addition + | Impeller | Chopper |
| Dose | Batch | materials taken | kneading | speed | speed |
| strength | No. | for granulation) | (minutes) | (rpm) | (rpm) |
| 25 | 1 | 34 | 10 | 500 | 2000 |
| 25 | 2 | 28 | 14 | 500 | 2000 |
| 25 | 3 | 28 | 14 | 300 | 1000 |
| 25 | 4 | 28 | 14 | 300 | 1000 |
| 25 | 5 | 22 | 18 | 200 | 0 |
| 25 | 6 | 22 | 6 | 200 | 2000 |
| 15 | 7 | 24 | 24 | 203 | 1500 |
| 50 | 8 | 24 | 26 | 203 | 1500 |

The dissolution rate of each of the Formulation B batches was then measured using the basket method in pH 4.5 acetate buffer as described in Example 9. The porosity of the blend of Formulation B batches, in terms of medium pore diameter, cumulative pore volume, or cumulative pore volume, was also measured using the methodology set out in US Pharmacopeia (USP 39-NF 34) Chapter <267> “Porosimetry by Mercury Intrusion”. The results of these measurements are set out in Table 12 below. The relative dissolution profiles between the six different 25 mg Formulation B batches are shown in FIG. 7.

TABLE 12
Porosimetry data of the blend filled into different capsule strengths
of Formulation B and corresponding dissolution results

| Pressure range (X ± 10%- | | | 397-0.01 | 49-0.2 | 49-0.2 | 49-0.01 |
|---|---|---|---|---|---|---|
| Y ± 10%) in MPa | | | | | | |
| Corresponding pore diameter | | | 0.004-130 | 0.03-9 | 0.03-9 | 0.03-130 |
| range (μm)* | | | | | | |
| Dose | | % Cumulative | Cumulative | Median pore | Cumulative | Cumulative |
| strength | Batch | release at 15 | pore volume | diameter | pore volume | pore volume |
| (mg) | No. | minutes | [mm³/g] | [μm] | [mm3/g] | [mm3/g] |
| 25 | 1 | 34 | 505 | 0.9 | 196 | 484 |
| 25 | 2 | 36 | 571 | 0.6 | 115 | 547 |
| 25 | 3 | 58 | 802 | 1.5 | 231 | 778 |
| 25 | 4 | 59 | 776 | 1.5 | 202 | 758 |
| 25 | 5 | 90 | 913 | 2.2 | 283 | 888 |
| 25 | 6 | 95 | 950 | 1.9 | 284 | 932 |
| 15 | 7 | 88 | 724 | 2.6 | 159 | 710 |
| 50 | 8 | 79 | 779 | 1.6 | 205 | 764 |

*Pore diameter is calculated using the Washburn equation with surface tension of 0.48 N/m and contact angle of 140° in the temperature range of 20 to 25° C.

The data demonstrates that the use of 34% water during wet granulation and a high impeller speed of 500 rpm leads to overgranulation and, thereby, lower blend porosity. This is reflected in the relatively poor dissolution profile of Batch 1 of the 25 mg does strength Formulation B. Similarly, the use of 28% water during wet granulation, a high 500 rpm impeller speed in conjunction with 14 minutes granulation time, leads to overgranulation and lower blend porosity. This is reflected in the relatively poor dissolution profile of Batch 2. In contrast, the use of 28% water, a 300 rpm impeller speed, and 14 minute granulation time avoided overgranulation, improved the degree of blend porosity, and resulted in a much enhanced dissolution profile for Batches 3 and 4. Moreover, the use of 22% water, a 200 rpm impeller speed and an 18 or 6 minute granulation time, led to a further improvement in blend porosity and dissolution profile for Batches 5 and 6.

These data demonstrate that the degree of blend porosity is a crucial factor in determining the dissolution rate of the Compound 1 formulation.

### Example 11: Relative Bioavailability of Experimental Formulation and Formulations A and B

Human in vivo exposure to drug substance was tested in an open-label, randomized, single dose cross-over PK study in healthy adult male subjects to assess the relative bioavailability of three different formulations of Compound 1.

### Study Design

This was an open-label, randomized, 3-period, single dose crossover study to assess the relative bioavailability of 3 different Compound 1 formulations in healthy adult male subjects. A total of 16 subjects were randomized in a 1:1 ratio into 2 treatment sequences: Cohort 1 (8 subjects) or Cohort 2 (8 subjects). Screening occurred from Day −28 to Day −2. Baseline 1 occurred on Day −1, Baseline 2 was on Day 21, and Baseline 3 was on Day 42. The treatment arms are summarised in Table 13 below.

In Treatment Period 1, on Day 1;
subjects in Cohort 1 received Compound 1 FB 50 mg
subjects in Cohort 2 received Compound 1 FA 50 mg,
followed by a 3-week washout period (Days 2 to 21) and Baseline 2 on Day 21.

In Treatment Period 2, the order of treatment was reversed, i.e. on Day 22
subjects in Cohort 1 received Compound 1 FA 50 mg
subjects in Cohort 2 received Compound 1 FB 50 mg,
followed by a 3-week washout period (Days 23 to 42) and Baseline 3 on Day 42.

At the end of Treatment Period 2, an interim analysis was performed for data collected in Treatment Periods 1 and 2 while Treatment Period 3 continued.

In Treatment Period 3, Cohort 1 and Cohort 2 were assigned to 2 parallel sub-cohorts. On Day 43,
subjects in Cohort 1 were assigned to receive either Compound 1 FB 10 mg (4 subjects) or Compound 1 EF 50 mg (4 subjects)
subjects in Cohort 2 were assigned to receive either Compound 1 FB 10 mg (4 subjects) or Compound 1 EF 50 mg (4 subjects),
followed by a 3-week assessment period (Days 44 to 63).

TABLE 13
Treatment arms of relative bioavailability study

| Treatment arm | Dose level |
|---|---|
| Formulation A (FA) | 50 mg (25 mg HGC × 2) |
| Formulation B (FB) | 50 mg (25 mg HGC × 2) |
| Experimental Formulation (EF) | 50 mg (25 mg HGC × 2) |
| Formulation B (FB) | 10 mg (10 mg HGC × 1) |

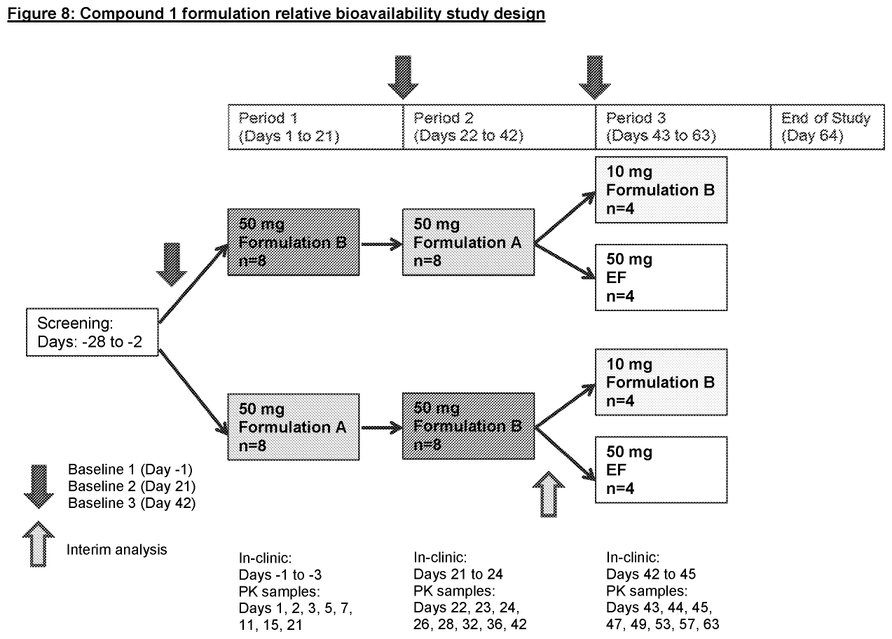

The design of the relative bioavailability study is shown in FIG. 8.

### PK Assessments

### Drug Concentration Measurements

All blood samples (3 mL) were taken by either direct venipuncture or an indwelling catheter inserted in a forearm vein. At specified time points, blood sample were collected in tubes with a specific anticoagulant K₃EDTA. Immediately after each tube of blood was drawn, it was gently inverted several times to ensure the mixing of tube contents. Tubes were stored upright in a test tube rack surrounded by ice until centrifugation. Within 30 minutes of collection, the sample was centrifuged between 3° C. and 5° C. for 10 minutes at approximately 2000 g (or samples were centrifuged at room temperature if tubes were placed on ice immediately after processing). Immediately after centrifugation, the whole supernatant was transferred into uniquely labeled 1.8 mL polypropylene tubes. The tubes were immediately frozen over solid carbon dioxide (dry ice) then kept frozen at ≤−65° C. pending analysis.

The frozen plasma samples were thawed at room temperature and sonicated before aliquoting. A volume of 25 μL plasma samples (standard, QC, blank, study sample) was transferred into a 1.00 mL V-bottom 96 square-well plate. A volume of 225 μL acetonitrile containing 0.025% TFA and containing [¹³C2D₃] Compound 1 at 6.00 ng/mL or 225 μL of acetonitrile containing 0.025% TFA for the blank samples was added into each well. The well plate was mixed on the shaker for about 5 min at 1000-1500 rpm and then centrifuged at 5650 g for 10 minutes at approximately 10° C. The plate was finally placed in the chilled auto-sampler and 3 μL of the supernatant was analyzed by liquid chromatography-tandem mass spectrometry (LC-MS/MS) in MRM positive mode using ESI as the ionization technique. Compound 1 was quantified over the range from 1.00 ng/mL (LLOQ) to 1000 ng/mL (ULOQ) using 0.025 mL of human plasma.

### PK Results

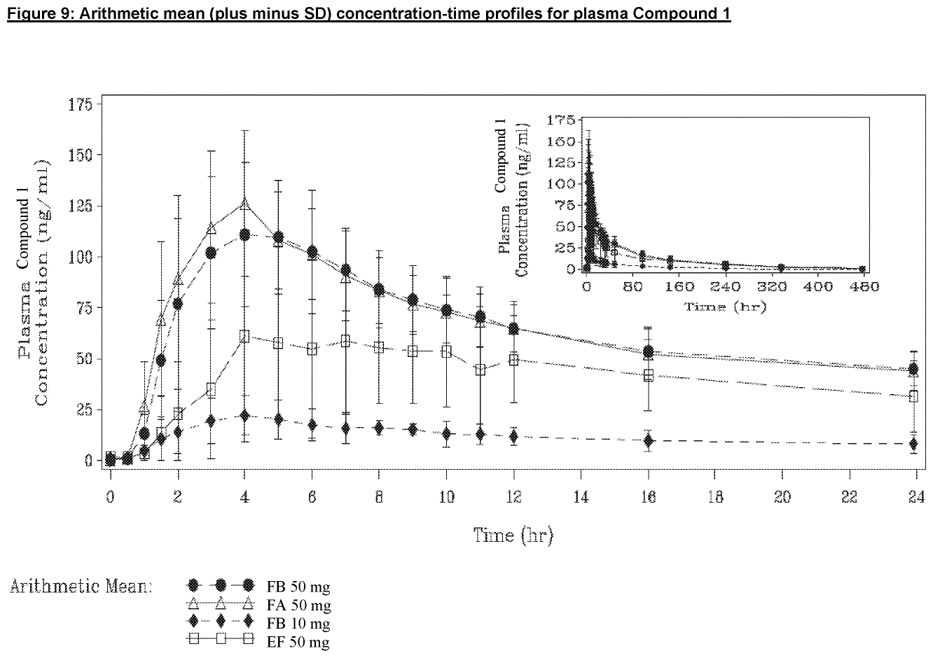

Plasma PK profiles of the formulations tested in the relative bioavailability study are shown in FIG. 9 and Table 14. Formulations A and B were comparable after single oral administration of 50 mg Compound 1 with respect to bioavailability as shown by similar AUCinf and Cmax values. The EF showed delayed Tmax (5.0 hours versus 4.0 hours) whereas mean Cmax and AUCinf of Compound 1 for the EF formulation were significantly lower compared to the corresponding values for Formulations A and B, illustrative of the relatively poor bioavailability of the EF. The lower Cmax and AUCinf for EF is in-line with the slower in vitro dissolution profile of the EF at pH 4.5 observed in comparison to Formulations A and B (See Example 9). These results demonstrate the significantly improved bioavailability of Formulations A and B and the bio-relevance of the pH 4.5 acetate buffer dissolution condition.

TABLE 14
Predose-corrected PK parameters

| | | AUCinf** | | AUClast | | Cmax | | Tmax | T½*** |
|---|---|---|---|---|---|---|---|---|---|
| Treatment | Statistics | (ng*h/mL) | | (ng*h/mL) | | (ng/mL) | | (h) | (h) |
| FB 50 mg | n* | 15 | | 15 | | 15 | | 15 | 15 |
| [N = 15] | Mean (SD) | 5750 | (1710) | 5550 | (1660) | 121 | (33.2) | 4.00 | 89.2 (20.1) |
| | Geo-mean | 5550 | (27.7) | 5340 | (28.0) | 118 | (25.8) | [2.00; 6.00] | 87.0 (23.7) |
| | (CV %) | | | | | | | | |
| FA 50 mg | n* | 15 | | 15 | | 16 | | 16 | 15 |
| [N = 16] | Mean (SD) | 5500 | (1280) | 5290 | (1260) | 128 | (34.9) | 4.00 | 87.1 (25.1) |
| | Geo-mean | 5370 | (23.2) | 5160 | (23.7) | 124 | (26.2) | [1.50; 4.00] | 84.2 (27.0) |
| | (CV %) | | | | | | | | |
| FB 10 mg | n* | 6 | | 7 | | 7 | | 7 | 6 |
| [N = 7] | Mean (SD) | 930 | (263) | 763 | (320) | 22.4 | (7.39) | 4.00 | 52.1 (20.4) |
| | Geo-mean | 888 | (37.6) | 690 | (56.2) | 21.5 | (31.0) | [3.00; 10.0] | 48.9 (39.7) |
| | (CV %) | | | | | | | | |
| EF 50 mg | n* | 6 | | 7 | | 7 | | 7 | 6 |
| [N = 7] | Mean (SD) | 3960 | (2060) | 3610 | (2000) | 74.3 | (44.1) | 5.00 | 68.2 (16.1) |
| | Geo-mean | 3540 | (54.7) | 3210 | (55.0) | 65.1 | (58.8) | [4.00; 12.0] | 66.8 (22.8) |
| | (CV %) | | | | | | | | |

FA = Formulation A
FB = Formulation B
EF = Experimental Formulation

### Example 12: First-in-Human Study Demonstrating Lack of Food Effect

This study was a randomised, double-blind, placebo-controlled, single and multiple ascending oral dose study to primarily assess the safety and tolerability as well as the pharmacokinetics and pharmacodynamics of Compound 1 in healthy adult and elderly subjects. Food effect was studied in 10 subjects after administration of 75 mg Formulation A together with a high fat meal and under fasting condition. The rate of absorption of Compound 1 was not affected when taken together with a high fat meal as compared to intake of Compound 1 in a fasting state, as median Tmax was 4.04 and 3.50 h, respectively. Food intake increased the Cmax and AUC0-72 h slightly, since the geometric mean for the ratio fed/fasted was 1.11 and 1.10 respectively.

### Example 13: In Human Study of Pharmacokinetics of Compound 1 when Given Alone and in Combination with the Strong CYP3A4 Inhibitor Itraconazole or the Strong CYP3A4 Inducer Rifampicin

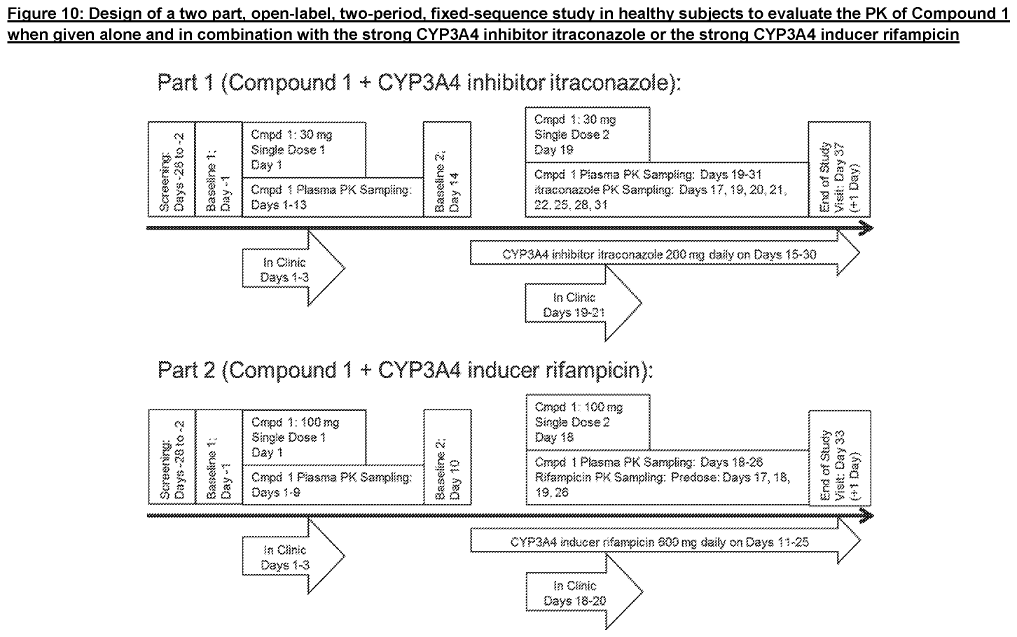

In a drug-drug interaction (DDI) study in healthy volunteers, the effect of a strong CYP3A4 inhibitor (itraconazole) and a strong CYP3A4 inducer (rifampicin) on the PK of Compound 1 was evaluated. The DDI study design is outlined in FIG. 10. Itraconazole, at a dose of 200 mg q.d., increased mean AUC of Compound 1 2-3-fold and mean Cmax of Compound 1 by 25%, when given together with Compound 1 as compared to when Compound 1 was given alone (Table 15). Rifampicin, at a dose of 600 mg q.d., decreased mean AUC of Compound 1 5-6-fold and mean Cmax of Compound 1 2.5-fold, when given together with Compound 1 as compared to when Compound 1 was given alone (Table 16). In conclusion, the effect of a strong CYP3A4 inducer and a strong CYP3A4 inhibitor on Compound 1 exposure in a Phase 1 study has shown that CYP3A4 is of major importance for the elimination of Compound 1 and that the effects of co-treatment with a strong CYP3A4 inhibitor or inducer need to be taken into account when administering a formulation comprising Compound 1.

TABLE 15
Pharmacokinetic results - Statistical analysis of the effect of
itraconazole on the plasma PK parameters of Compound 1: Compound
1 30 mg SD + itraconazole 200 mg QD vs Compound 1 30 mg SD

| | | | Adjusted | Geometric | |
|---|---|---|---|---|---|
| Parameter | | | geometric | mean ratio | 90% CI |
| [Unit] | Treatment | n* | mean | (Test/Reference) | for ratio |
| AUCinf | Cmpd 1 30 mg SD | 17 | 3560 | 3.05 | [2.91, 3.20] |
| (ng*hr/mL) | Cmpd 1 30 mg SD + | 17 | 10900 | | |
| | Itraconazole 200 mg QD | | | | |
| AUClast | Cmpd 1 30 mg SD | 17 | 3150 | 2.20 | [2.11, 2.30] |
| (ng*hr/mL) | Cmpd 1 30 mg SD + | 17 | 6930 | | |
| | Itraconazole 200 mg QD | | | | |
| Cmax | Cmpd 1 30 mg SD | 17 | 74.1 | 1.23 | [1.18, 1.29] |
| (ng/mL) | Cmpd 1 30 mg SD + | 17 | 91.3 | | |
| | Itraconazole 200 mg QD | | | | |

n* = number of subjects with non-missing values.

An ANOVA model with fixed effects for treatment and subject was fitted to each log-transformed PK parameter. Results were back transformed to obtain ‘Adjusted geo-mean’, ‘Geo-mean ratio’ and ‘90% Cl’.

TABLE 16
Pharmacokinetic results - statistical analysis of the effect
of rifampicin on the plasma PK parameters of Compound 1: Compound
1 100 mg SD + rifampicin 600 mg QD vs Compound 1 100 mg SD

| | | | Adjusted | Geometric | |
|---|---|---|---|---|---|
| Parameter | | | geometric | mean ratio | 90% CI |
| [Unit] | Treatment | n* | mean | (Test/Reference) | for ratio |
| AUCinf | Cmpd 1 100 mg SD | 13 | 10200 | 0.172 | [0.152, 0.194] |
| (ng*hr/mL) | Cmpd 1 100 mg SD + | 13 | 1750 | | |
| | Rifampicin 600 mg QD | | | | |
| AUClast | Cmpd 1 100 mg SD | 13 | 8560 | 0.196 | [0.176, 0.219] |
| (ng*hr/mL) | Cmpd 1 100 mg SD + | 13 | 1680 | | |
| | Rifampicin 600 mg QD | | | | |
| Cmax | Cmpd 1 100 mg SD | 13 | 222 | 0.414 | [0.365, 0.470] |
| (ng/mL) | Cmpd 1 100 mg SD + | 13 | 92.2 | | |
| | Rifampicin 600 mg QD | | | | |

n* = number of subjects with non-missing values.

An ANOVA model with fixed effects for treatment and subject was fitted to each log-transformed PK parameter. Results were back transformed to obtain ‘Adjusted geo-mean’, ‘Geo-mean ratio’ and ‘90% Cl’.

### REFERENCES

International Conference on Harmonisation (ICH) of Technical Requirements for Registration of Pharmaceuticals for Human Use: Stability Testing of New Drug Substances and Products Q1A9R2); Step 4 version dated 6 Feb. 2003 (the “ICH Q1A Guidance”).
Amidon G L et al. (1995) A theoretical basis for a biopharmaceutic drug classification: the correlation of in vitro drug product dissolution and in vivo bioavailability. Pharm. Res.; 12(3):413-420.
Heimbach T et al. (2013) Case Studies for Practical Food Effect Assessments across BCS/BDDCS Class Compounds using In Silico, In Vitro, and Preclinical In Vivo Data. AAPS J.; 15(1):143-158.
Klein S (2010) The Use of Biorelevant Dissolution Media to Forecast the In Vivo Performance of a Drug. AAPS J.; 12(3):397-406.
Kramp V P, Herrling P, (2011) List of drugs in development for neurodegenerative diseases: Update June 2010. Neurodegener. Dis.; 8(1-2):44-94.
Sheskey P J et al. (2017) Handbook of Pharmaceutical Excipients, 8th Revised Edition.
Sinko P J, Martin A N (2011) Martin's physical pharmacy and pharmaceutical sciences: Physical chemical and biopharmaceutical principles in the pharmaceutical sciences (6th Ed.). Philadelphia: Lippincott Williams & Wilkins.
Sperling R A et al. (2011) Toward defining the preclinical stages of Alzheimer's disease: Recommendations from the National Institute on Aging and the Alzheimer's Association workgroup. Alzheimers Dement.; 7(3):1-13.
Stahl H, Wermuth C (2011) Pharmaceutical Salts: Properties, Selection, and Use, 2^{nd} Revised Edition

All references, e.g., a scientific publication or patent application publication, cited herein are incorporated herein by reference in their entirety and for all purposes to the same extent as if each reference was specifically and individually indicated to be incorporated by reference in its entirety for all purposes.

1. A pharmaceutical composition comprising the drug substance N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide and having a blend with:
(i) a median pore diameter of at least 1 μm, as determined by mercury porosimetry, within the 0.03 to 9 μm pore diameter range;
(ii) a cumulative pore volume of at least 200 mm³/g, as determined by mercury porosimetry, within the 0.03 to 9 μm pore diameter range; or
(iii) a cumulative pore volume of at least 600 mm³/g, as determined by mercury porosimetry, within the 0.004 to 130 μm pore diameter range.

2. A pharmaceutical composition comprising the drug substance N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide wherein subsequent to single dose oral administration to a human subject the plasma Cmax value of the drug substance measured in ng/mL is a function of the drug substance dose in mg multiplied by a factor of 2.4, within a +/−range defined by the drug substance dose in mg multiplied by a factor of 0.7, when the pharmaceutical composition comprises greater than or equal to 10 mg of drug substance or less than or equal to 50 mg of drug substance.

3. A pharmaceutical composition comprising the drug substance N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide and having a dissolution profile wherein at least 40% of the cumulative drug substance release is observed after 15 minutes dissolution testing using the basket apparatus method described in US Pharmacopeia Chapter <711> and the following testing parameters:
Dissolution medium: acetate buffer pH 4.5;
Apparatus 1: 100 rpm;
Total Measurement Time: 60 minutes; and
Temperature: 37±0.5° C.

4. A pharmaceutical composition comprising the drug substance N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide wherein said drug substance is present within the pharmaceutical composition in an amount greater than 7% w/w.

5. A pharmaceutical composition comprising the drug substance N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide and a sugar alcohol.

6. A pharmaceutical composition comprising the drug substance N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide;
(i) a sugar alcohol;
(ii) starch or cellulose; and
(iii) hydroxypropyl cellulose or hydroxypropyl methylcellulose.

7. The pharmaceutical composition according to claim 5 wherein the sugar alcohol is mannitol.

8. The pharmaceutical composition according to claim 1 wherein the median pore diameter is at least 1.4 μm within the 0.03 to 9 μm pore diameter range.

9. The pharmaceutical composition according to claim 1 wherein the cumulative pore volume is at least 220 mm³/g within the 0.03 to 9 Lm pore diameter range.

10. The pharmaceutical composition according to claim 1 wherein the cumulative pore volume is at least 700 mm³/g within the 0.004 to 130 μm pore diameter range.

11. The pharmaceutical composition according to claim 3 wherein at least 60% of the cumulative drug substance release is observed after 15 minutes.

12. The pharmaceutical composition according to claim 4 comprising:
(i) 1 to less than 25 mg of drug substance wherein said drug substance is present within the pharmaceutical composition in an amount greater than 7% w/w; or
(ii) 25 to 50 mg of drug substance wherein said drug substance is present within the pharmaceutical composition in an amount greater than 17% w/w.

13. The pharmaceutical composition according to any one of claims 1 to 12 comprising;
(i) mannitol;
(ii) cellulose; and
(iii) hydroxypropyl methylcellulose.

14. The pharmaceutical composition according to any one of claims 1 to 12 comprising:
(i) between 25 and 50% w/w mannitol;
(ii) between 10 and 60% w/w cellulose;
(iii) between 1 and 10% w/w low-substituted hydroxypropyl cellulose;
(iv) between 1 and 5% w/w hydroxypropyl methylcellulose;
(v) between 0.1 and 1% w/w talc; and
(vi) between 0.5 and 3% w/w sodium stearyl fumarate.

15. The pharmaceutical composition according to claim 14 wherein the pharmaceutical composition comprises 15 or 50 mg of drug substance.

16. The pharmaceutical composition according to claim 15 wherein the drug substance Compound 1 is in crystalline Form A and wherein crystalline Form A has an X-ray powder diffraction pattern with at least three peaks having angle of refraction 2 theta (θ) values selected from 10.7, 14.8, 18.7, 19.5 and 21.4° when measured using CuKα radiation, wherein said values are plus or minus 0.2° 2θ.

17. A process for the preparation of a pharmaceutical composition comprising the drug substance N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide wherein the drug substance is co-milled with a sugar alcohol.

18. A process according to claim 17 wherein the sugar alcohol is mannitol.
